SEQUENCE LISTING (2 sequences)

SEQ ID NO: 1 - DNA, 19 bp, Artificial sequence
  Synthetic peptide
accacccttcgaaccacac

SEQ ID NO: 2 - DNA, 22 bp, Artificial sequence
  Synthetic peptide
tttgtaaaacggactgttagag

(19) United States
(12) Patent Application Publication
(10) Pub. No.: US 2009/0137789 A1
(43) Pub. Date: May 28, 2009

(54) Oligonucleotide Probes

(76) Inventors: Giovanna Barbarella, Bologna (IT); Massimo Luigi Capobianco, Bologna (IT); Giovanna Sotgiu, Ozzano Dell'Emilia (IT); Massimo Zambianchi, Cesena (IT); Fabio Aruffo, Fara San Martino (IT); Mario Benzi, Casalecchio Di Reno (IT); Marina Naldi, Pianoro (IT); Umberto Negri, Bologna (IT)

Correspondence Address: MARSHALL, GERSTEIN & BORUN LLP, 233 SOUTH WACKER DRIVE, 6300 SEARS TOWER, CHICAGO, IL, 60606-6357, US

(21) Appl. No.: 11/667,610

(22) PCT Filed: Nov. 10, 2005
(86) PCT No.: PCT/IB05/03369

§ 371 Date: Aug. 11, 2008

(30) Foreign Application Priority Data
Nov. 11, 2004 (IT) BO2004A000697

(51) Int. Cl. C07H 21/04; C07D 409/14

(52) U.S. Cl. 536/24.3; 548/527

(57) ABSTRACT

Fluorescent oligonucleotide probe having a general formula (I), wherein Onu represents an oligonucleotide residue, Thioₙ represents a fluorescent oligothiophene containing n thiophenic rings, n being an integer lower than 8, L representing a binder conceived to maintain Thioₙ mobile in relation to Onu in a manner so that Thioₙ can perform its fluorescent action correctly, and Onu can perform its hybridization action.

(I)=Onu-L-Thioₙ.

### TECHNICAL FIELD

The present invention relates to oligonucleotide probes, methods for their synthesis and intermediate compound beacons for said methods.

### BACKGROUND ART

Numerous biotechnological applications use oligonucleotides because of their intrinsic capacity to selectively bond to complementary strands or to particular duplex using Watson and Crick or Hoogsteen type hydrogen bonds. These applications are largely facilitated because of the possibility of specifically recognizing oligonucleotides used as an “instrument” by the oligonucleotides present in the analytic sample. In order to do this, the oligonucleotide is “marked” with an easily recognizable group such as a radioactive isotope, for example, or a dye, or a fluorescent group. The oligonucleotide marked in this manner is referred to as an oligonucleotide probe.

Among main probe uses, is the localization of the probes themselves in the various cellular districts by means of fluorescent microscopy, and the detection of complementary sequences with “molecular beacons” for example (probes that quench their own fluorescence signal where are self-associated, but by hybridizing with the oligonucleotide of the target sequence they undergo conformational change returning to become fluorescent once more, revealing the presence of the target compound) when searching for alien DNA (viral or transgenic DNA) or modified DNA, or for amplification applications through PCR (polymerase chain reaction) etc.

For some time, fluorophors derived from fluorescein, have been used as fluorescent beacons for biological systems such as proteins, membranes and also nucleotides. In particular, it is usual practice to incorporate fluorescent beacons derived from fluorescein with oligonucleotides in order to obtain oligonucleotide probes.

Oligonucleotide probes derived from fluorescein present the following problems: relatively high “photobleaching”, (fluorescence loss); fluorescence dependent on pH, whose fluorescence is considerably reduced under a level of pH 7; a relatively wide emission spectrum that limits applications where different color emission is necessary (Handbook of fluorescent probes and research products, 9th Ed; Molecular Probes Inc.: Eugene, Oreg., 2002; Ch 1 Section 1.1 pg 47).

Certain applications where different color probes are required are: flow cytometry, DNA sequencing, fluorescent microscopy, etc.

### DISCLOSURE OF INVENTION

An object of the present invention is to create oligonucleotide probes that are free of the problems described above. Further aims of the present invention are to supply synthesis methods for said oligonucleotide probes using compound beacons as well as realizing the compound beacons themselves.

According to the present invention oligonucleotide probes are created according to the general formula (I)

Onu-L-Thioₙ (I)

as recited in the first-claim and, preferably, in any one of the claims dependent either directly or indirectly contingent on the first claim.

According to the present invention compounds are also provided according to the general formula III:

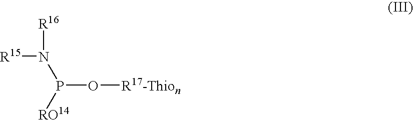

as recited in claim 16, and preferably, in any one of the subsequential claims dependent either directly or indirectly contingent to claim 16.

According to the present invention compounds are also provided according to the general formula V:

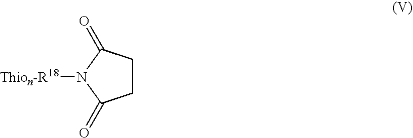

as recited in claim 30, and preferably, in any one of the subsequential claims dependent either directly or indirectly on claim 30.

According to the present invention compounds are also provided according to the general formula VI:

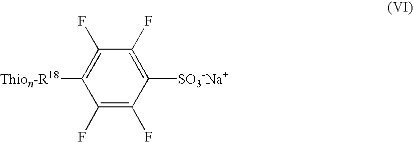

as recited in claim 38, and preferably, in any one of the subsequential claims dependent either directly or indirectly on claim 38.

According to the present invention compounds are also provided according to the general formula VII:

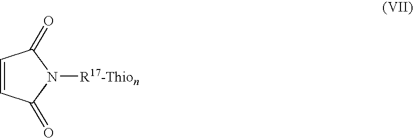

as recited in claim 46, and preferably, in any one of the claims dependent either directly or indirectly on claim 46.

According to the present invention methods are also provided, as described in the appended claims for the preparation of the probes according to the general formula I bonding oligonucleotides to compounds according to the general formulas III, V, VI and VII.

### BRIEF DESCRIPTION OF THE DRAWINGS

The invention is described below with reference to the appended drawings which relate to not limiting embodiments, wherein:

FIG. 1 shows an absorption spectrum: the wave length is shown in the X-axis in nm, and the absorbency is shown in the Y-axis;

FIG. 2 shows an emission spectrum: the wave length is shown in the X-axis in nm, and the emission intensity is shown in the Y-axis;

FIG. 3 shows an absorption spectrum: the wave length is shown in the X-axis in nm, and the absorbency is shown in the Y-axis;

FIG. 4 shows an emission spectrum: the wave length is shown in the X-axis in nm, and the emission intensity is shown in the Y-axis;

FIG. 5 shows an NMR spectrum;

FIG. 6 shows an absorption spectrum: the wave length is shown in the X-axis in nm, and the absorbency is shown in the Y-axis;

FIG. 7 shows an emission spectrum: the wave length is shown in the X-axis in nm, and the emission intensity is shown in the Y-axis;

FIG. 8 shows an absorption spectrum: the wave length is shown in the X-axis in nm, and the absorbency is shown in the Y-axis; and

FIG. 9 shows an absorption spectrum: the wave length is shown in the X-axis in nm, and the emission intensity is shown in the Y-axis;

### BEST MODE FOR CARRYING OUT THE INVENTION

The definitions of the various chemical moieties will be introduced in the next few paragraphs, and are to be understood as being applied in the same manner throughout the whole text, including the claims, unless another definition is specifically set out.

The term “oligonucleotide” refers to a single strand DNA or RNA fragment composed of at least two nucleotides. In particular, an oligonucleotide comprises between two and two hundred nucleotides, preferably between 20 and 140 nucleotides and even more preferably between 20 and 60 nucleotides. This definition includes modified oligonucleotides, commonly used in biotechnological applications (such as phosphorothioates methylphosphonates, 2′-O-alkyl-RNA, 5′-alkylpyrimidine for example) whose synthesis occurs with the simple extension of standard protocols and whose transformation into fluorescent probes can be performed through simple extension of the methods described in the present text.

The term “thiophenic ring” refers to an aromatic ring with 5 members, wherein one of the members is a sulphur atom; optionally, the aromatic ring can have one or more substituents; optionally the sulphur can be present in the form of an oxide. Examples of thiophenic rings are the following:

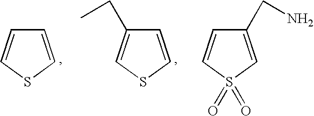

The term “oligothiophene” refers to one or more thiophenic rings connected to each other in linear or branched mode, or fused together. Below are some examples of oligothiophenes:

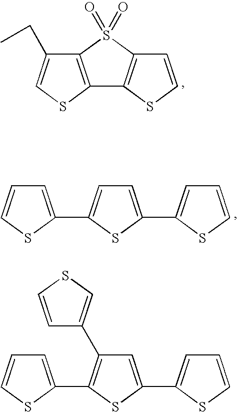

The term “halogen” refers to a radical selected from the group consisting of: chlorine, fluorine, bromine, iodine.

The term “alkyl Cₓ-C_{y}” refers to at linear or branched monovalent alkyl group presenting a minimum of x carbon atoms and a maximum of y carbon atoms. This term is exemplified by groups such as methyl, ethyl, n-propyl, isopropyl, n-butyl, isobutyl, ter-butyl and n-hexyl.

The term “alkenyl Cₓ-C_{y}” refers to a linear or branched monovalent alkenyl group presenting a minimum of x carbon atoms and a maximum of y carbon atoms and at least one unsaturated site. This term is exemplified by groups such as vinyl (—CH═CH₂) andn-2-propenyl(allyl, —CH₂CH═CH₂).

The term “alkynyl Cₓ-C_{y}” refers to a linear or branched monovalent alkynyl group presenting a minimum of x carbon atoms and a maximum of y carbon atoms and at least one doubly unsaturated site. This term is exemplified by groups such as: ethynyl (—C≡CH) and propargyl (—CH₂C≡CH).

The term “cycloalkyl Cₓ-C_{y}” refers to a saturated carbocyclic group presenting a minimum of four and a maximum of eight carbon atoms, and having a single ring (for example cyclohexyl) or condensed multiple rings.

The term “aliphatic Cₓ-C_{y}” refers to a monovalent group “cycloalkyl Cₓ-C_{y}”, “alkyl Cₓ-C_{y}”, “alkenyl Cₓ-C_{y}” or “alkynyl Cₓ-C_{y}”.

The term “alkylene Cₓ-C_{y}” refers to a bivalent group “cycloalkyl Cₓ-C_{y}”, “alkyl Cₓ-C_{y}”, “alkenyl Cₓ-C_{y}” or “alkynyl Cₓ-C_{y}”. As an option, the alkylene can contain one or more ether atoms, in particular N, O and S, each of which is bonded to two carbon atoms. This term is exemplified by groups such as —CH₂—CH₂—, —CH═CH—, —C≡C—, —CH₂CH═CH—, —CH₂—CH₂—O—CH₂—, —CH₂—S—CH₂— and —CH₂—N—(CH₃)—CH₂—CH₂—.

The term “saturated alkylene Cₓ-C_{y}” refers to a bivalent group “cycloalkyl Cₓ-C_{y}”, “alkyl Cₓ-C_{y}”. This term is exemplified by groups such as —CH₂—CH₂—, —(CH₂)₃—.

The term “arylic” or “aryl” refers to a phenyl group or a system of fused bicyclic or tricyclic rings, wherein at least one of the fused rings is a phenylic group. Examples of bicyclic and tricyclic systems are naphtalene and phenantrene. Optionally, the acrylic groups can be substituted by one to five substituents. In particular the substituents may be selected, each independently from one other, from the group consisting of: halogens, aliphatics C₁-C₁₂, carbonyls, alkanoyls, alkoxys, hydroxy (—OH), mercapto (—SH), alkylsulfonyls, alkylsulfanyls, cyano (—CN), nitro (—NO₂), alkylcarboxys, amines (—NH₂), alkylamines.

The term “alkanoyl” refers to an aliphatic group C₃-C₁₂ bonded to the remaining part of the molecule through a carbonyl group.

The term “alkoxy” “refers to an aliphatic group C₁-C₃₂ bonded to the remaining part of the molecule through an atom of oxygen.

The term “alkylsulfanyl”, refers to an aliphatic group or a substituted aliphatic group bonded to the remaining part of the molecule through a sulphur atom.

The term “alkylsulfonyl” refers to an aliphatic group C₁-C₁₂ bonded to the remaining part of the molecule through a group —SO₂—.

The term “alkylcarboxy” refers to an alcoxy group bonded to the remaining part of the molecule through a carbonyl group.

The term “alkylammine” refers to one or two aliphatic groups C₁-C₁₂ bonded to the remaining part of the molecule through a nitrogen atom.

The term “substituted aliphatic Cₓ-C_{y}” refers to an aliphatic group Cₓ-C_{y} substituted by one or more substituents selected from a group composed of: halogens, carboxy groups, carbonyl groups, alkanoyl groups, alkoxy groups, hydroxyls, sulphydryl groups (—SH), alkoxy groups, alkylsuphonyl groups, alkylsulphanyl groups, cyano groups, nitro groups, alkylcarboxy, with the proviso that the number of substituents is always lower than the number of hydrogen atoms of the corresponding non-substituted aliphatic group.

According to a first aspect of the invention presented herein, an oligonucleotide probe having general formula (I) is provided:

Onu-L-Thioₙ (I)

wherein Onu refers to an oligonucleotide residue; each Thio refers to a respective thiophenic ring, independently from the other Thio; Thioₙ refers to a fluorescent oligothiophene containing n thiophenic rings; n is an integer lower than eight; L refers to at binder conceived to maintain Thioₙ mobile in relation to Onu so that Thioₙ is able to perform its fluorescent action correctly and Onu is able to hybridize freely with a complementary sequence; with the proviso that if n is equal to one, the sulphur of the thiophenic ring is bonded to two oxygens.

According to a further aspect of the invention presented herein, a beacon compound having a general formula III is provided:

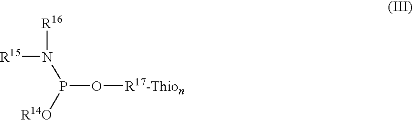

wherein R¹⁴ is a protecting group of the phosphite ester and is selected so that R¹⁴ is removable through the action of a 30% aqueous ammonia solution; R¹⁵ and R¹⁶ being selected so that NR¹⁵R¹⁶ is removable through the action of the weak acids, in particular tetrazole; each Thio refers to a respective thiophenic ring, independently from the other Thio; Thioₙ refers to a fluorescent oligothiophene containing n thiophenic rings; R¹⁷ being selected from the group consisting of: —R¹²— and —R⁹—C(O)—; R¹² being selected from the group consisting of: an alkylene C₁-C₉, —R⁹—Si(R¹⁰)(R¹¹)—; R¹⁰ and R¹¹ representing, each independently from each other, a respective alkyl C₁-C₈, aryl, alkenyl C₂-C₈, alkynyl C₂-C₈; R⁹ representing an alkylene C₁-C₈.

According to certain embodiments, R¹⁵ and R¹⁶ are selected, each independently from each other, from the group consisting of: alkyl C₁-C₁₂, aryl, cycloalkyl C₅-C₁₀; R¹⁵ and R¹⁶ can be linked so that, together with N, they form a 5-6 member heterocyclic ring. Preferably, R¹⁵ and R¹⁶ represent, each independently from each other, a saturated alkyl C₁-C₃. Even more preferably, R¹⁵ and R³⁶ represent, each independently from each other, a saturated alkyl C₃. Particularly preferable are the embodiments wherein R¹⁵ and R¹⁶ represent, each, a respective isopropyl group.

Preferably, R¹⁴ is selected from the group consisting of: —(CH₂)₂CN and —CH₃.

According to a further aspect of the invention presented herein a beacon compound is described presenting a general formula V:

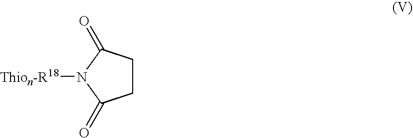

each Thio refers to a respective thiophenic ring, independently from the other Thio; Thioₙ refers to a fluorescent oligothiophene containing n thiophenic rings; R¹⁸ being selected from the group consisting of: —C(O)—O—, —R¹²—C(O)—O—, —C(O)—R⁹—C(O)—O—; R¹² being selected from the group consisting of: an alkylene C₁-C₉, —R⁹—Si(R¹⁰)(R¹¹)—; R¹⁰ and R¹¹ representing, each independently from each other, a respective alkyl C₁-C₈, aryl, alkenyl C₂-C₈, alkynyl C₂-C₈; R⁹ representing an alkylene C₁-C₈.

Preferably the compound has the following formula:

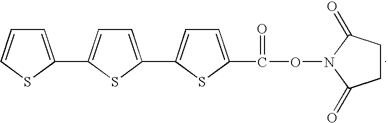

According to a further aspect of the invention presented herein a beacon compound presenting a general formula VI is provided:

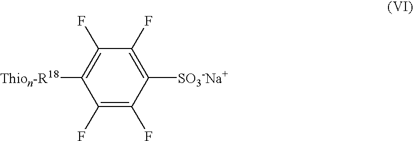

each Thio refers to a respective thiophenic ring, independently from the other Thio; Thioₙ refers to a fluorescent oligothiophene containing n thiophenic rings; R¹⁸ being selected from the group consisting of: —C(O)—O—, —R¹²—C(O)—O—, —C(O)—R⁹—C(O)—O—; R¹² being selected from the group consisting of: an alkylene C₁-C₉, —R⁹—Si(R¹⁰)(R¹¹)—; R¹⁰ and R¹¹ representing, each independently from each other, a respective alkyl C₁-C₈, aryl, alkenyl C₂-C₈, alkynyl C₂-C₈; R⁹ representing an alkylene C₁-C₈.

Preferably, the compound has the following formula:

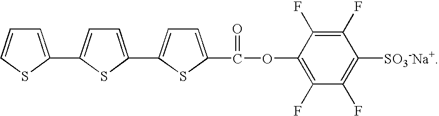

According to a further aspect of the invention presented herein a beacon compound is described presenting a general formula VII:

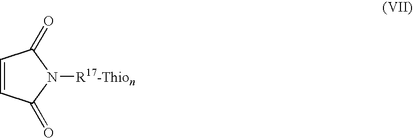

each Thio refers to a respective thiophenic ring independently from the other Thio; Thioₙ refers to a fluorescent oligothiophene containing n thiophenic rings; R¹⁷ being selected from the group consisting of: —R¹²— and —R⁹—C(O)—; R¹² being selected from the group consisting of: an alkylene C₁-C₉, —R⁹—Si(R¹⁰)(R¹¹)—; R¹⁰ and R¹¹ representing, each independently from each other, an alkyl C₁-C₈, aryl, alkenyl C₂-C₈, alkynyl C₂-C₈; R⁹ representing an alkylene C₁-C₈. Preferably, R¹² represents a saturated alkylene C₂-C₉ and R⁹ represents a saturated alkylene C₁-C₈. Even more preferably, R³⁷ represents a saturated alkylene C₂-C₃.

The above-described compounds can be obtained by using the following general procedure. It should be understood that where typical or preferred experimental conditions are described (this refers to: temperature, reaction times, reactant moles, solvents etc.) other conditions could be used. Optimal reaction conditions can vary according to the particular reactants or solvents used.

According to a further aspect of the invention presented herein a method for the preparation of an oligonucleotide probe is also provided, having the general formula I as defined above, wherein L is selected from a group composed of —O—P(O)₂—O—R¹²—, —O—P(O)₂—O—R⁹—C(O)—; the method providing for a conjugation phase, wherein a compound having the general formula III as defined above is bonded to an oxygen in position 5′ of a nucleoside of an oligonucleotide residue Onu; during said conjugation phase NR¹³R¹⁶ being removed and P being oxidized; a removal phase, that occurs in basic conditions and during which R¹⁴ is removed. R¹², R⁹, R¹⁵, R¹⁶ and R¹⁴ are defined as above.

Preferably, the conjugation phase occurs in a moderately acidic environment, in particular in the presence of tetrazole. Typically the conjugation phase is performed in the typical conditions of an automatic oligonucleotide synthesizer.

It is important to emphasize that, in this manner, using a normal oligonucleotide it is possible to incorporate the oligothiophene in position 5′ of the oligonucleotide residue Onu.

According to certain preferred embodiments, the compound having general formula III as defined above is obtained by means of nucleophilic sunstitution, wherein a first reactant, which has general formula Thioₙ-R¹⁷—O⁻, is made to react with a second reactant having general formula IV:

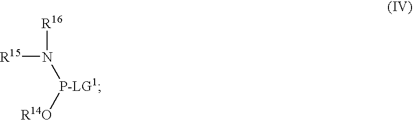

LG¹ being a leaving group. R¹⁷ is defined as above. Preferably, LG¹ is a leaving group selected from the group consisting of: halogen and NR¹⁵R¹⁶.

Preferred embodiments of the above-described method can be schematically represented as follows:

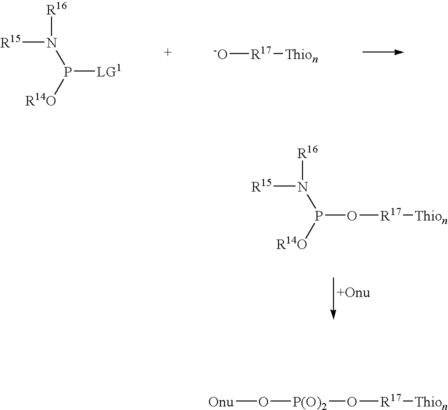

According to a further aspect of the invention presented herein a method is provided for the synthesis of an oligonucleotide probe, having general formula I as defined above, wherein L is selected from the group consisting of R⁸—NH— (O)C—R⁹—C(O)—, —R⁸—NH— (O)C—R¹²—, —R⁸—NH— (O)C—; the method comprising a conjugation phase wherein a compound having the general formula V as defined above, is made to react with prearranged oligonucleotide residue having the formula Onu-R⁸—NH₂, wherein Onu represents an oligonucleotide residue; R⁸ representing an alkylene C₁-C₈. Preferably, R⁸ is a hexyl group. R⁹ and R¹² are defined as above.

Preferred embodiments of the above-described method can be schematically represented as follows:

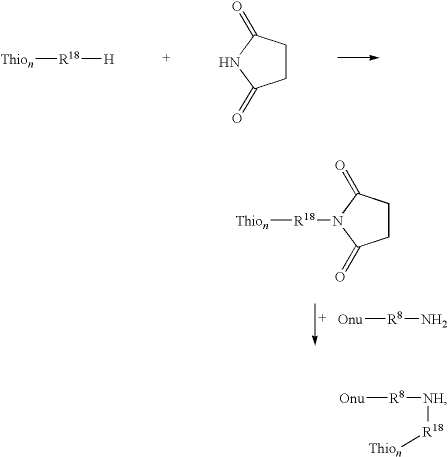

wherein R¹⁸ is defined as above.

According to a further aspect of the invention presented herein a method is provided for the synthesis of an oligonucleotide probe, presenting the general formula I as defined above, wherein L is selected from the group consisting of R⁸—NH— (O)C—R⁹—C(O)—, —R⁸—NH—(O)C—R¹²—, —R⁸—NH— (O)C—; the method comprising a conjugation phase, wherein a compound having the general formula VI as defined above is made to react with a prearranged oligonucleotide residue having the formula Onu-R⁸—NH₂, wherein Onu represents an oligonucleotide; R⁸ representing an alkylene C₁-C₈. Preferably, R⁸ is a hexyl group.

Preferred embodiments of the above-described method can be schematically represented as follows:

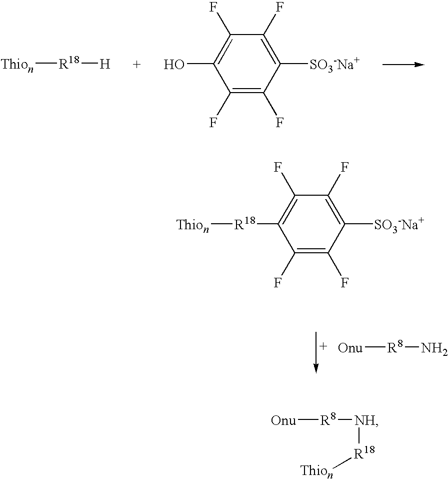

wherein R¹⁸ is defined as above.

According to a further aspect of the invention presented herein a method is provided for the preparation of an oligonucleotide probe, presenting the general formula I as defined above, wherein L is selected from the group consisting of;

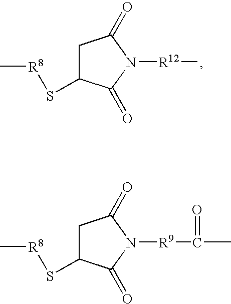

the method providing for a conjugation phase, wherein a compound having the general formula VII as defined above is made to react with a prearranged oligonucleotide residue having the formula Onu-R⁸—SH, wherein Onu represents an oligonucleotide residue; R⁸ representing an alkylene C₁-C₈. Preferably, R⁸ is a hexyl group.

Preferably, the method comprises a nucleophilic substitution phase, wherein a first reactant, which is selected from the group consisting of Thio, —R¹²—O⁻ Thioₙ-C(O)—R⁹—O⁻, is made to react with

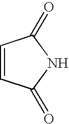

in order to obtain the compound presenting the general formula VII as defined above. Preferably the nucleophilic substitution occurs in the presence of PPh₃, (CH₃)₃COH and CH₃ CH₂C(O)N₂C(O)CH₂CH₃.

Preferred embodiments of the above-described method can be schematically represented as follows:

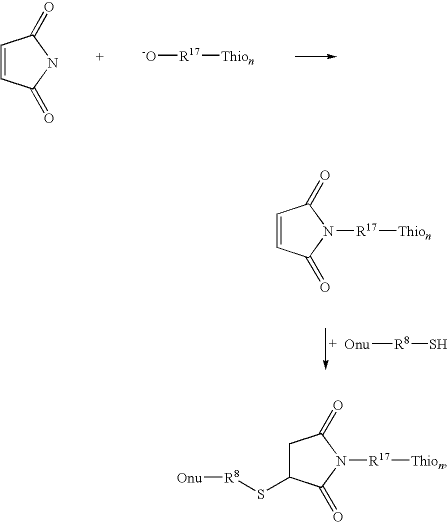

wherein R¹⁷ is defined as above. A possible alternative strategy for the synthesis of probes having the general formula I, wherein L is selected among —R⁸—NH—C(S)—NH—R¹²— and —R⁸—NH—C(S)—NH—R⁹—C(O)—, can be schematically represented as follows:

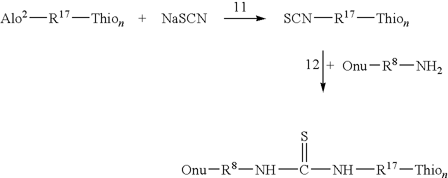

R⁹, R¹², R¹⁷ and R⁸ are defined as above. Alo² represents a halogen. Step 11 is a nucleophilic substitution and preferably occurs in acetone. Step 12 occurs, preferably in DMF/H₂O.

Possible alternative strategies for the synthesis of probes presenting the general formula I, wherein L is selected from —R⁸—S—, —R⁸—S—R³²— and —R⁸— S—R⁹—C(O)—, are schematically represented as follows:

Onu-R⁸—SH+X—R¹²-Thioₙ→Onu-R⁸—S—R¹²-Thioₙ

Onu-R⁸—SH+X—R⁹—C(O)-Thioₙ→Onu-R⁸—S—R⁹—C(O)-Thioₙ

Onu-R⁸—SH+X-Thioₙ→Onu-R⁸—S-Thioₙ

Onu-R⁸—X+HS—R¹²-Thioₙ→Onu-R⁸—S—R¹²-Thioₙ

Onu-R⁸—X+HS—R⁹—C(O)-Thioₙ→Onu-R⁸—S—R⁹—C(O)-Thioₙ

Onu-R⁸—X+HS-Thioₙ→Onu-R⁸—S-Thioₙ

X represents a bromine, an iodine or a chlorine. R⁸, R⁹ and R¹² are defined as above.

A further strategy to incorporate an oligothiophene Thioₙ in an Onu oligonucleotide residue consists in bonding the ologothiophene Thioₙ to a single nucleoside and then modifying the nucleoside in order to obtain a beacon wherein the nucleoside with phosphoroamidite is bonded to the oligothiophene Thioₙ (phosphoroamidite-nucleoside-Thioₙ). As an alternative option, the nucleoside can be appropriately modified in order to present in position 5′, the hydroxy protected by a protecting group —PG, which is removable in an acid environment; in this case it is possible to incorporate the beacon in position 3′, in position 5, and in an intermediate position of the oligonucleotide. Wherever the beacon is to be incorporated in position 3′, the beacon will be attached to an automatic synthesizer column, then followed by the synthesis of the oligonucleotide. Wherever the beacon is to be incorporated in position 5′, incorporation will be carried out on completion of the synthesis (3′→5′) of the oligonucleotide. Wherever the beacon is to be incorporated in position 5′, incorporation will be carried out at an appropriate moment during the synthesis (3′→5′) of the oligonucleotide.

A possible synthesis strategy for the reagents Thioₙ-R¹⁸—H is schematically represented as follows:

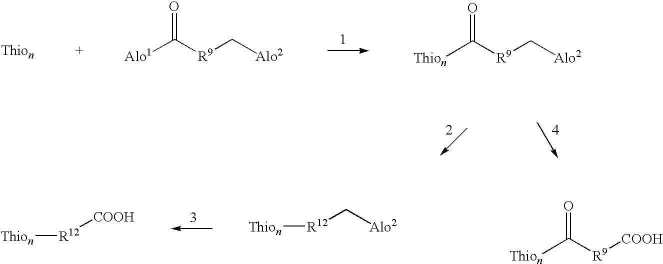

R¹² represents an alkylene C₂-C₉. R⁹ and R¹⁸ are defined as above. Alo¹ and Alo² represent respective halogens, each independently from each other. Step 1 is an acilation and preferably occurs in the presence of AlCl₃. Step 2 is a reduction and preferably occurs in the presence of AlCl₃ and LiAlH₄. Steps 4 and 3 are oxidations and involve treating the halogen derivates with KCN, in order to obtain a nucleophilic substitution with cyano, which through hydrolysis leads to carboxylic acids. Step 3 is schematically shown as follows:

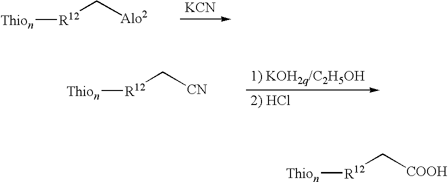

According to a further approach, a possible strategy for synthesizing reagent Thioₙ-R¹⁸—H is schematically shown as follows:

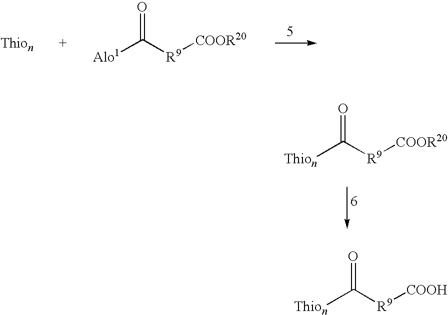

R⁹ and R¹⁸ are defined as above. Alo¹ is defined as above, R²⁰ represents an alkyl. Step 5 is an acilation and preferably, occurs in the presence of AlCl₃. Step 6 is a hydrolysis and preferably, occurs in EtOH in presence of NaOH.

A possible strategy for synthesizing reagent ⁻O—R¹⁷-Thioₙ is schematically represented as follows:

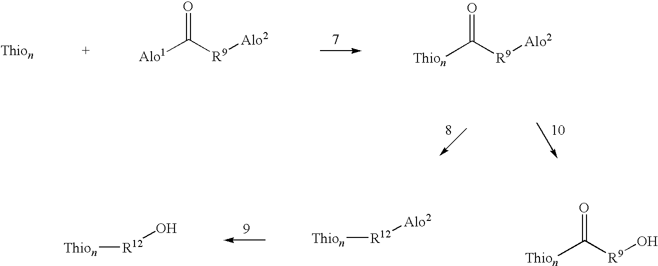

R¹² represents an alkylene C₂-C₉. R⁹, R¹⁷, Alo¹ and Alo² are defined as above. Step 7 is an acilation and preferably, occurs in the presence of AlCl₃. Step 8 is a reduction and preferably, occurs in the presence of AlCl₃ and LiAlH₄. Steps 9 and 10 are nucleophilic substitutions and occur preferably, in a basic environment; even more preferably, steps 9 and 10 occur in the presence of N-Methylpyrrolidone (NMP).

A possible strategy for reactant synthesis Alo²—R¹²-Thioₙ, wherein R¹² represents —R⁹—Si(R¹⁰)(R¹¹)—, is schematically represented as follows:

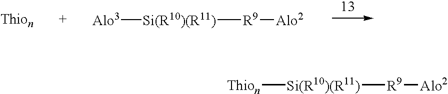

R⁹, R¹⁰, R¹¹ and Alo² are defined as above. Alo³ represents a halogen. Step 13 occurs preferably in presence of LDA (lithiumdiisopropylamine) o of n-butylthio.

A possible strategy for the synthesis of oligothiophene Thioₙ is schematically represented as follows:

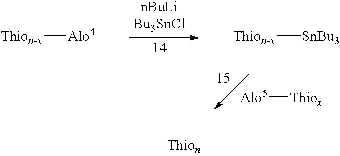

Thio and Thioₙ are defined as above. Alo⁴ and Alo⁵ are, each independently from each other, halogens. Preferably, Alo⁴ and Alo⁵ represent, each, a respective bromine atom. Preferably, Step 14 is carried out in ethylic ether. Preferably, Step 15 is carried out in toluene. Even more preferably Step 15 carried out in presence of Pd(Ph₃As)₄ (F. Effenberger, F Wurthner, F steybe J. Org: Chem., 1995, 60, 2082-2091; G. Barbarella, M. Zambianchi, G. Sotgiu, A. Bongini Tetrahedron 1997, 53, 9401-9406).

A further possible strategy for oligothiophene Thioₙ synthesis is also described in Jong, F. D.; Janssen, M. J. J. Org. Chem. 1971, 36, 1645-1648 and is shown in the example below:

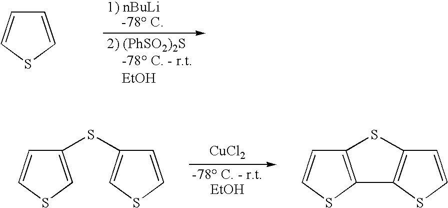

A possible method for oxidizing the sulphur of a thiophenic ring involves using an oxidizing agent, in particular mCPBA. Even more preferably the reaction occurs in CH₂Cl₂.

With reference to what has been described above, according to preferred embodiments, independently from the other Thio, each Thio represents a respective thiophenic, ring presenting the general formula II:

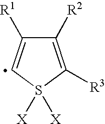

wherein X is chosen from a free lone pair of S electrons and an oxygen radical; R¹, R², R³ are selected, each independently from one other, from the group consisting of: hydrogen, halogen, alkyl, C₁-C₁₂, aromatic, —CN, —NO₂, -Thioₘ, —NR⁴R⁵, —SR⁴, —R⁵—SR⁴, —OR⁴, —R⁵—OR⁴, —C(O)R⁶, —NC(O)R⁶, —O—PG, —R⁷—O—PG; wherein R⁴ and R⁵ are selected, each independently from each other, from the group consisting of hydrogen, alkyl C₁-C₁₂, aromatic, Thioₘ; R⁶ is selected from the group consisting of hydrogen, halogen, alkyl C₁-C₁₂, aromatic, Thioₘ; m is an integer lower than n; —PG is a protecting group of hydroxy that is removable in an acidic environment; with the proviso that, if n is equal to 1, X represents an oxygen radical; each Thio group can be fused with 1 or 2 other Thio; R⁷ is a saturated alkylene C₁-C₈. Preferably, the aforesaid aromatic groups are acrylic groups.

Preferably, n is lower than 5. According to a preferred embodiment, R¹, R² and R³ are selected, each one in a manner separate from one other, from the group consisting of: alkyl C₁-C₈, —R⁵—S—R⁴, hydrogen, —SR⁴, —R⁷—O—PG; wherein R⁷, R⁴ and R⁵ represent each one in a manner separate from one other, a saturated alylene C₁—Ce; —PG is defined as above and represents a protecting group of the hydroxy that is removable in an acidic environment.

Even more preferably, R¹, R², R³ are, each one in a manner separate from one other, selected from the group consisting of: hydrogen, —SR⁴, —R⁷—O—PG. Particularly preferable are the embodiments wherein R¹, R², R³ represent, each, a respective hydrogen.

Preferably, X represents a lone pair of electrons of S. Preferably, —PG is selected from the group consisting of: dimethoxytrityl, monomethoxytrityl, methoxy ethoxy methyl (MEM), methoxy methyl (MOM).

Particularly preferable are the embodiments, wherein Thioₙ represents:

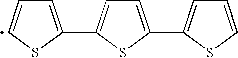

With reference to what has been described above, according to preferred embodiments, L is selected from the group consisting of: alkylene C₂-C₈, —R⁸—C(O)—, —R⁸—O—C(O)—, —R⁸—C(O)—R⁹—, —R′—O—C(O)—R⁹—, —R⁸—O—R⁹—C(O)—, —R⁸—O—R¹²—, —R⁸—S—, —R⁸—S—R⁹—C(O)—, —R⁸—S—R¹², —R⁸—NH—R⁹—C(O)—, —R⁸—NH—R¹²—, —R⁸—Si (R¹⁰)(R¹¹)—, —O—P(O)₂—O—R¹²—, —O—P(O)₂—O—R⁹—C(O)—, —R⁸—NH—(O)C—R⁹—C(O)—, —R⁸—NH—(O)C—R¹²—, —R⁸—NH— (O)C—, —R⁸—NH—C(S)—NH—R¹²—, —R⁸—NH—C(S)—NH—R⁹—C(O)—,

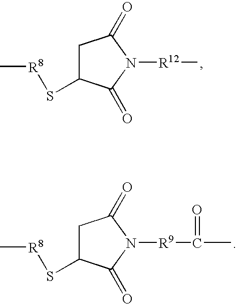

Preferably, L is selected from the group consisting of: saturated alkylene C₂-C₈, —R⁸—C(O)—, —R⁸—O—C(O)—, R⁸—C(O)—R⁹—, —R⁸—O—C(O)—R⁹—, —R⁸—O—R⁹—C(O)—, —R⁸—O—R¹²—, —R⁸—S—R⁹—C(O)—, —R⁸—S—R¹²—, —R⁸—NH—R⁹—C(O)—, —R⁸—NH—R¹²—, —R⁸—Si(R¹⁰)(R¹¹)—, —O—P(O)₂—O—R¹²—, —O—P(O)₂—O—R⁹—C(O)—, —R⁸—NH—(O)C—R⁹—C(O)—, —R⁸—NH— (O)C—R¹²—, —R⁸—NH—(O)C—, —R⁸—NH—NH—C(S)—NH—R¹²—, —R⁸—NH—C(S)—NH—R⁹—C(O)—,

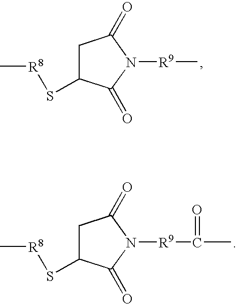

Even more preferably, L is selected from the group consisting of: —R⁸—S—R⁹—C(O)—, —R⁸—S—R¹²—, —O—P(O)₂—O—R¹²—, —O—P(O)₂—O—R⁹—C(O)—, —R⁸—NH—(O)C—R⁹—C(O)—, —R⁸—NH—(O)C—R¹²—, —R⁸—NH— (O)C—, —R⁸—NH—C(S)—NH—R¹²—, —R⁸—NH—C(S)—NH—R⁹—C(O)—,

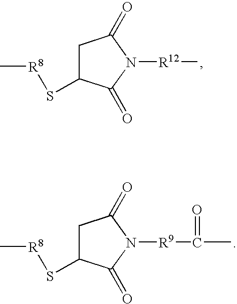

Even more preferably, L is selected from the group consisting of: —O—P(O)₂—O—R¹²—, —O—P(O)₂—O—R⁹—C(O)—, —R⁸—NH—(O)C—R⁹—C(O)—, —R⁸—NH—(O)C—R¹²—, —R⁸—NH— (O)C—;

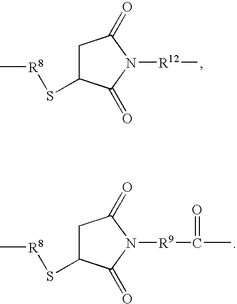

Particularly preferable are the embodiments wherein L is selected from the group consisting of: —R⁸—NH— (O)C—, —O—P(O)₂—O_R¹²—.

With reference to what has been described above, according to preferred embodiments R¹⁰ and R¹¹ represent, each independently from each other, a respective alkyl C₁-C₆. Preferably, R¹⁰ and R¹¹ represent, each independently from each other, a respective saturated alkyl.

According to preferred embodiments, R⁸ and R⁹ represent, each independently from each other, a respective alkylene C₁-C₆. Preferably, R⁸ and R⁹ represent, each independently from each other, a respective saturated alkylene. Even more preferably, R⁸ and R⁹ represent, each independently from each other, a respective linear alkylene. Particularly preferable are the embodiments wherein R⁸ represents a hexyl.

Preferably, R¹² is selected from the group consisting of: alkylene C₂-C₉, —R⁹—Si (R¹⁰)(R¹¹)—. More preferably, R¹² represents an alkylene C₂-C₇. Even more preferably, R¹² represents an alkylene C₂-C₃. According to preferred embodiments, R¹² represents a saturated and preferably linear alkylene.

From the descriptions provided above it is obvious that the compounds presenting the general formulas III, V, VI and VII can be used not only as beacons for oligonucleotide probes but also as beacons for any other type of application including pharmacology.

Oligonucleotide probes having the general formula I, have the following advantages compared to known oligonucleotide probes:
they are relatively very stable compounds even when irradiated for long periods of time, and basically are not subject to photobleaching (in other words, they do not loose their fluorescence);
their properties are independent of the solution pH;
they have Stokes shift (in other words, the difference between the emitted wave length and that of the absorbed light) relatively high;
they have relatively high quantum yields;
the molecular hybridization methods can be standardized because the compound beacons belong to a homogeneous class of molecules; and
by varying the oligothiophene, the colors of the light emitted by the probes can be varied making applications possible wherever different colored emissions are required.

Further characteristics of the present invention will be made clear from the following description of certain examples provided simply as illustrations and not to be considered in any way limiting.

The examples from 1 to 7 are schematically illustrated in the scheme 1 shown below wherein R represents a hydrogen:

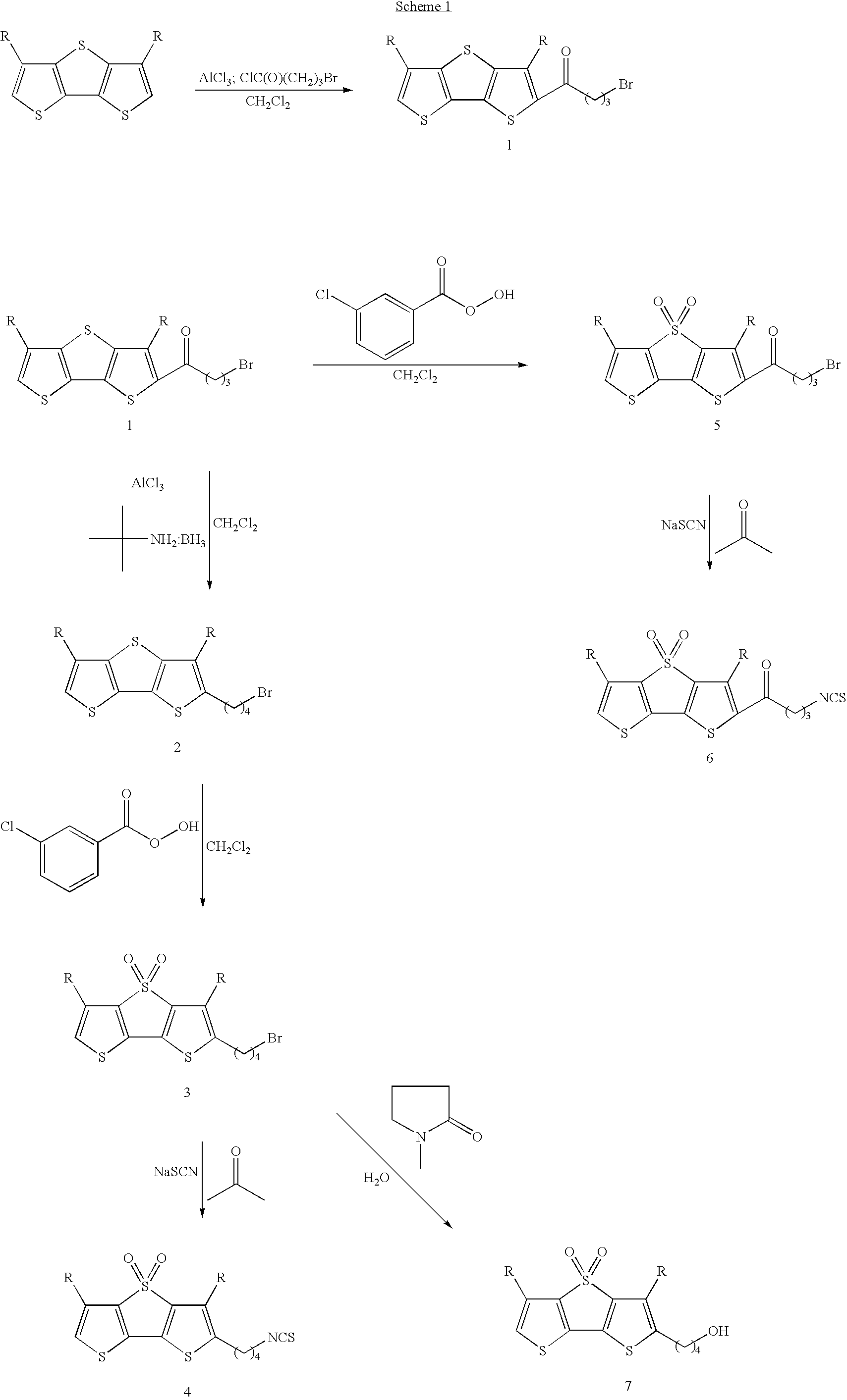

### EXAMPLE 1

### 4-bromo-1-dithien[3,2-b; 2′,3′-d]thien-2-yl-butane-1-one (1)

Bromobutyrylchloride (2.35 mmol, 0.27 mL) is added to a solution of aluminium chloride (2.82 mmol, 375 mg) in 20 mL of methylene chloride at 0° C. The mixture is stirred for an hour and added a drop at a time to a solution of dithien[3,2-b; 2′,3′-d]thiophene (2.35 mmol, 460 mg) dissolved in 25 mL of methylene chloride at 0° C. The reaction mixture is left to be mixed at room temperature overnight and is later quenched with a solution of hydrochloric acid 0.1M. The product is extracted using ether and methylene chloride and the organic phases are anhydrified with anhydrous sodium sulphate. The solvent is eliminated using a rotavapor and the residue purified through crystallization from pentane. 649 mg of a light green powder is obtained (Yield 80%): mp 104° C.; MS m/e 346 (M^{+}); FTIR (neat) v_{CO} 1649 cm⁻¹; ¹H NMR (CDCl₃, TMS/ppm) δ 7.972 (s, 1H), 7.535 (d, ³J=5.2 Hz, 1H), 7.332 (d, ³J=5.2 Hz, 1H), 3.565 (t, 3J=6.4 Hz, 2H), 3.174 (t, ³J=6.8 Hz, 2H), 2.349 (m, 2H); ¹³C NMR (CDCl₃, TMS/ppm) δ 192.154, 145.437, 143.927, 141.453, 137.47, 131.056, 129.501, 126.253, 121.183, 36.978, 33.730, 27.409

### EXAMPLE 2

### 2-(4-bromo-butyl)-dithien[3,2-b;2′,3′-d]thiophene (2)

A solution of 4-bromo-1-dithien[3,2-b;2′,3′-d]thien-2-yl-butane-1-one (1) (0.0030 mol, 1 g) in 20 mL of methylene chloride is added a drop at a time to a solution of aluminium chloride (0.015 mol, 2.04 g) and borane tributylamine (0.031 mol, 2.66 g) in 30 mL of methylene chloride at 0° C. The reaction mixture is left to be stirred for 4 hours at room temperature and is then quenched with a solution of hydrochloric acid 0.1M. the product is extracted with diethyl ether and methylene chloride, the organic phase anhydrified with sodium sulphate and the solvent eliminated with a rotavapor. The residue is purified through flash-chromatography on aluminia using the mixture of pentane: methylene chloride 9:1, as an eluent, to obtain 1.20 g of yellow oil product (Yield 71%): MS m/e 332 (M^{+}); ¹H NMR (CDCl₃, TMS/ppm) δ 7.308 (d, ³J=4.8 Hz, 1H), 7.264 (d, ³J=4.8 Hz, 1H), 6.991 (t, ⁴J=1.2 Hz, 1H), 3.442 (t, ³, J=6.4 Hz, 2H), 2.946 (t, ³J=6.4 Hz, 2H), 1.931 (m, 4H); ¹³C NMR (CDCl₃, TMS/ppm) δ 145.814, 140.714, 140.236, 131.091, 128.860, 125.119, 120.664, 117.933, 33.287, 31.722, 30.176, 29.918

### EXAMPLE 3

### 2-(4-bromo-butyl)-dithien[3,2-b;2′,3′-d]thiophene-4,4-dioxide (3)

A solution of m-chloro perbenzoic acid (5.438 mmol, 1.22 g), previously anhydrified with magnesium sulphate is added a drop at a time to a solution of 2-(4-bromo-butyl)-dithien[3,2-b;2′,3′-d]thiophene (2) (1.807 mmol, 0.6 g) in 25 mL of methylene chloride. The mixture is stirred overnight at room temperature, after which it is first washed with distilled water. KOH_{aq} at 10% and lastly NaHCO_{3aq} at 10% and extracted using methylene chloride. The organic phase is anhydrified on sodium sulphate, the solvent eliminated using the rotavapor and the residue purified using flash-chromatography on aluminia and using as an eluent the mixture ether: methylene chloride:ethyl acetate 6:1:3. 436 mg of a yellow solid are obtained (Yield 66%): mp 110° C.; MS m/e 364 (M^{+}); FTIR (neat) v_{SO2} 1307, 1139 cm⁻¹; ¹H NMR (CDCl₃, TMS/ppm) δ7.306 (d, ³J=5.2 Hz, 1H), 7.197 (d, ³J=5.2 Hz, 1H), 6.928 (t, ⁴J=1.2 Hz, 1H), 3.434 (t, ³J=6.4 Hz, 2H), 2.871 (t, ³J=8.4 Hz, 2H), 1.902 (m, 4H); ¹³C NMR (CDCl₃, TMS/ppm) δ 150.566, 142.606, 142.097, 136.489, 133.362, 128.923, 120.173, 117.153, 32.895, 31.551, 29.707, 29.707

### EXAMPLE 4

### 2-(4-isothiocyanate-butyl)-dithien[3,2-b;2′,3′-d]thiophene-4,4-dioxide (4)

A solution of 2-(4-bromo-butyl)-dithien[3,2-b;2′,3′-d]thiophene-4,4-dioxide (3) (0.137 mmol, 50 mg) and sodium thiocyanate (2.747 mmol, 222.5 mg) is placed in a 5 mL wheaton V-Vial in 5 mL of distilled acetone. The mixture is stirred strongly at 200° C. for 4 hours. After cooling at room temperature, the synthesis crude is filtered on a silica plug to remove excess sodium salt, and is then purified through crystallization from toluene/pentane to produce 40 mg (Yield 85%) of a yellow ochre solid: MS m/e 341 (M^{+}); FTIR (neat) v_{SO2} 1306, 1139 cm⁻¹; v_{NCS} 2149 cm⁻¹; λₘₐₓ (CH₂Cl₂)=364λₑₘ (CH₂Cl₂)=456 nm; ε (CH₂Cl₂)=8523 mol⁻¹*cm⁻¹; ¹H NMR (CDCl₃, TMS/ppm) δ 7.315 (d, ³J=5.2 Hz, 1H), 7.196 (d, ³J=5.2 Hz, 1H), 6.932 (t, ⁴J=1.2 Hz, 1H), 2.980 (t, ³J=6.8 Hz, 2H), 2.894 (t, ³J=7.6 Hz, 2H), 1.889 (m, 4H); ¹³C NMR (CDCl₃, TMS/ppm) δ 149.898, 142.643, 142.127, 136.352, 133.506, 129.074, 120.165, 117.258, 111.961, 33.456, 29.866, 29.525, 29.039

### EXAMPLE 5

### 4-(4,4-Dioxy-dithien[3,2-b;2′,3′-d]thien-2-yl)-butane-1-ol (7)

154 mg (0.424 mmol) of 2-(4-bromo-butyl)-dithien[3,2-b;2′,3′-d]thiophene-4,4-dioxide (3) is dissolved in 2 mL of N-Methylpyrrolidone (NMP) with the addition of 0.5 mL of distilled water. The mixture is brought to reflux and left to react for 12 hours. It is washed several times with water then with methylene chloride. The organic phase is anhydrified and the solvent eliminated by the rotavapor. The residue, a brown oil, is distilled to eliminate residue NMP and then purified using flash-chromatography on silica, eluting with ether:ethyl acetate: methylene chloride: 5:3:2. This produces 93 mg (Yield 74%) of a yellow crystalline solid: mp 115° C.; MS m/e 300 (M^{+}); FTIR (neat) v_{SO2} 1304, 1135 cm⁻¹, v_{OH} 3368 cm⁻¹, v_{CO} 1063 cm⁻¹; λₘₐₓ(CH₂Cl₂)=363 nm; λₑₘ(CH₂Cl₂)=457 nm ¹H NMR (CDCl₃, TMS/ppm) δ 7.293 (d, ³J=5.2 Hz, 1H), 7.188 (d, ³=5.2 Hz, 1H), 6.918 (t, ⁴J=1.2 Hz, 1H), 3.683 (t, 3J=6.4 Hz, 2H), 2.870 (t, ³J=8.4 Hz, 2H), 1.682 (m, 4H); ¹³C NMR (CDCl₃, TMS/ppm) δ 151.340, 142.545, 142.006, 136.610, 133.173, 128.817, 120.143, 116.993, 62.233, 31.680, 30.352, 27.628

### EXAMPLE 6

### 4-bromo-1-(4,4-dioxy-dithien[3,2-b;2′3′-d]thien-2-yl)-butane-1-one (5)

In solution of CH₂Cl₂ 4-bromo-1-dithien[3,2-b;2′,3′-d]thien-2-yl-butane-1-one(1) is made to react with mCPBA. A pale orange powder is obtained: mp 187° C.; MS m/e 378 (M^{+}); FTIR (neat) v_{SO2} 1303, 1141 cm⁻¹, v_{CO} 1646 cm⁻¹; λₘₐₓ (CH₂Cl₂)=374 nm; λₑₘ (CH₂Cl₂)=454 nm; ε (CH₂Cl₂)=14000 mol⁻¹*cm⁻1; ¹H NMR (CDCl₃, TMS/ppm) δ 7.769 (s, 1H), 7.543 (d, ³J=4.8 Hz, 1H), 7.298 (d, ³J=4.8 Hz, 1H), 3.117 (m, 4H), 2.316 (m, 2H); ¹³C NMR (CDCl₃, TMS/ppm) δ 191.067, 147.461, 144.957, 143.159, 141.914, 135.084, 132.420, 123.610, 120.703, 36.726, 32.886, 26.610

### EXAMPLE 7

### 4-isothiocyanate-1-(4,4-dioxy-dithien[3,2-b;2′,3′-d]thien-2-yl-butane-1-one (6)

4-bromo-1-(4,4-dioxy-dithien[3,2-b;2′,3′-d]thien-2-yl)-butane-1-one (5) is made to react with NaSCN in acetone. A red solid is obtained: mp 155° C.; MS m/e 355 (M^{+}); FTIR (neat) v_{SO2}1302, 1138 cm⁻¹, v_{CO} 1661 cm⁻¹, v_{NCS} 2152 cm⁻¹; λₘₐₓ (CH₂Cl₂)=480 nm; λₑₘ (CH₂Cl₂)=586 nm; ε (CH₂Cl₂)=44480 mol⁻¹*cm⁻¹; ¹H NMR (CDCl₃, TMS/ppm) δ 7.761 (s, 1H), 7.537 (d, ³J=5.2 Hz, 1H), 7.291 (d, ³J=5.2 Hz, 1H), 2.498 (s, 4H), 0.535 (s, 12H); ¹³C NMR (CDCl₃, TMS/ppm) δ 142.908, 142.840, 141.922, 137.588, 136.784, 134.318, 134.196, 133.225, 125.932, 125.864, 125.165, 115.611, 113.927, 18.634, −2.592

The examples from 8 to 10 are schematically illustrated in the scheme 2 shown below wherein R represents at hydrogen:

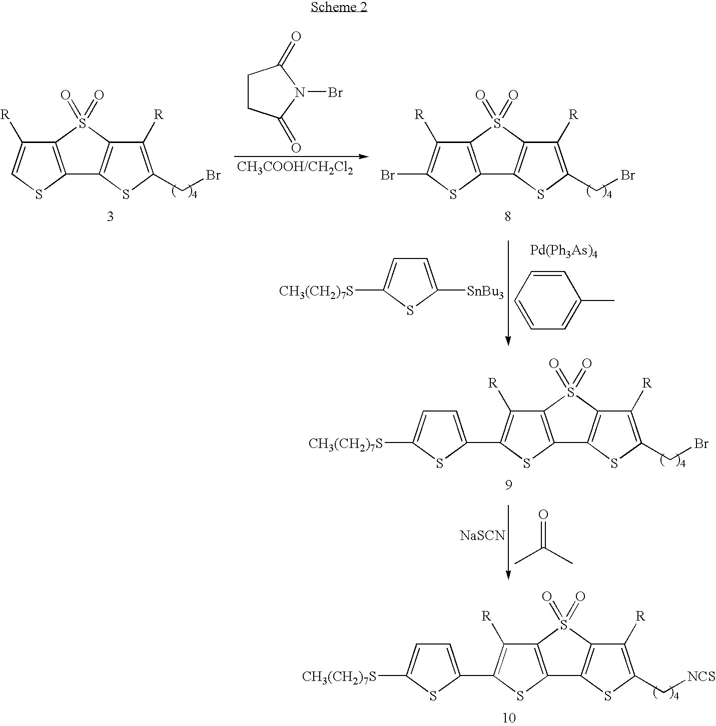

### EXAMPLE 8

### 2-(4-bromo-butyl)-6-bromo-dithien[3,2-b;2′,3′-d]thiophene-4,4-dioxide (8)

N-bromosuccinimide (0.493 mmol, 87.7 mg) is added in small doses to a solution of 2-(4-bromo-butyl)-dithien[3,2-b;2′,3′-d]thiophene-4,4-dioxide (0.448 mmol, 163 mg) dissolved in 20 mL of a solution 1:1 of methylene chloride: acetic acid at −20° C. and wrapped in tin foil to protect it from the light. The mixture is stirred at room temperature overnight and then quenched with water. The aqueous phase is extracted using methylene chloride, the organic phases are washed with KOH_{aq} at 10% k and NaHCO_{3aq} at 10%. Anhydrification is carried out with sodium sulphate, then the solvent is eliminated and the residue is crystallized from toluene/pentane to obtain 188 mg of yellow powder product (95% of Yield): mp 165° C.; MS m/e 442 (M^{+}); ¹H NMR (CDCl₃, TMS/ppm) δ 7.259 (s, 1H), 6.930 (t, ⁴J=1.2 Hz, 1H), 3.434 (t, ³J=6.4 Hz, 2H), 2.868 (t, ³J=8.0 Hz, 2H), 1.903 (m, 4H); ¹³C NMR (CDCl₃, TMS/ppm) δ 151.137, 141.431, 141.044, 136.331, 132.780, 122.558, 117.223, 115.773, 32.828, 31.545, 29.777, 29.686

### EXAMPLE 9

### 2-(4-bromo-butyl)-6-(5-octylsulfanyl-thien-2-yl)-dithien[3,2-b;2′,3′-d]thiophene-4,4-dioxide (9)

2-(4-bromo-butyl)-6-bromo-dithien[3,2-b;2′,3′-d]thiophene-4,4-dioxide (0.424 mmol, 188 mg) is dissolved in 5 mL of toluene followed by the addition of the catalyst generated in situ Pd(Ph₃As)₄ 0.011 mmol. The mixture is brought to reflux and the 2-tributylstannyl-5-octylsulfanyl-thiophene (0.488 mmol; 252 mg) is added a drop at a time, using a syringe. The mixture is left at reflux for another 5 hours, then the solvent is eliminated using the rotavapor and the residue purified using a chromatographic column on silica, with the mixture pentane:ethyl acetate: methylene chloride 6:3:1 as eluent, the product obtained recrystallizes from isopropylic alcohol to produce 194 mg (Yield 78%) of a micro-crystalline orange solid: mp 98° C.; MS m/e 590 (M^{+}); FTIR (neat) v_{SO2}1302, 1134 cm⁻¹; λₘₐₓ (CH₂Cl₂)=421 nm; λₑₘ (CH₂Cl₂)=550 nm; ¹H NMR (CDCl₃, TMS/ppm) δ 7.172 (s, 1H), 7.048 (d, ³J=3.6 Hz, 1H), 7.001 (d, ³J=3.6 Hz, 1H), 6.934 (t, ⁴J=0.8 Hz, 1H), 3.438 (t, ³J=6.4 Hz, 2H), 2.859 (m, 4H), 1.907 (m, 4H), 1.641 (m, 2H), 1.399 (m, 2H), 1.267 (bs, 8H), 0.875 (t, 6.8 Hz, 3H); ¹³C NMR (CDCl₃, TMS/ppm) δ 150.801, 142.461, 141.771, 141.194, 137.521, 137.225, 133.772, 133.469, 133.378, 125.091, 117.251, 115.574, 38.768, 32.864, 31.749, 31.574, 29.783, 29.723, 29.373, 29.131, 29.055, 28.410, 22.612, 14.082

### EXAMPLE 10

### 2-(4-isothiocyanate-butyl)-6-(5-octylsulfanyl-thien-2-yl)-dithien[3,2-b;2′,3′-d]thiophene-4,4-dioxide (10)

2-(4-bromo-butyl)-6-(5-octylsulfanyl-thien-2-yl)-dithien[3,2-b;2′,3′-d]thiophene-4,4-dioxide (9) is made to react with NaSCN in a solution of acetone. An orange solid is obtained: mp 102° C.; MS m/e 622 (M^{+}); FTIR (neat) vₛₒ₂1302, 1134 cm⁻¹, v_{NCS} 2154 cm⁻¹; λₘₐₓ (CH₂Cl₂)=421 nm; λₑₘ (CH₂Cl₂)=550 nm; ε(CH₂Cl₂)=28417 mol⁻¹*cm⁻¹; ¹H NMR (CDCl₃, TMS/ppm) δ 7.176 (s, 1H), 7.054 (d, 3J=4.0 Hz, 1H), 7.004 (d, ³J=3.6 Hz, 1H), 6.943 (t, ⁴J=1.2 Hz, 1H), 2.988 (t, ³J=6.8 Hz, 2H), 2.905 (t, ³J=7.6 Hz, 2H), 2.842 (t, ³J=7.2 Hz, 2H), 1.913 (m, 4H), 1.642 (m, 2H), 1.400 (m, 2H), 1.268 (bs, 8H), 0.875 (t, 6.8 Hz, 3H); ¹³C NMR (CDCl₃, TMS/ppm) δ 150.080, 142.529, 141.823, 141.337, 137.459, 137.308, 133.627, 133.551, 133.460, 125.135, 117.387, 115.573, 111.923, 38.767, 33.486, 31.740, 29.964, 29.562, 29.365, 29.122, 29.084, 29.054, 28.409, 22.611, 14.081

The examples from 11 to 13 are schematically illustrated in the scheme 3 shown below wherein R represents a hydrogen:

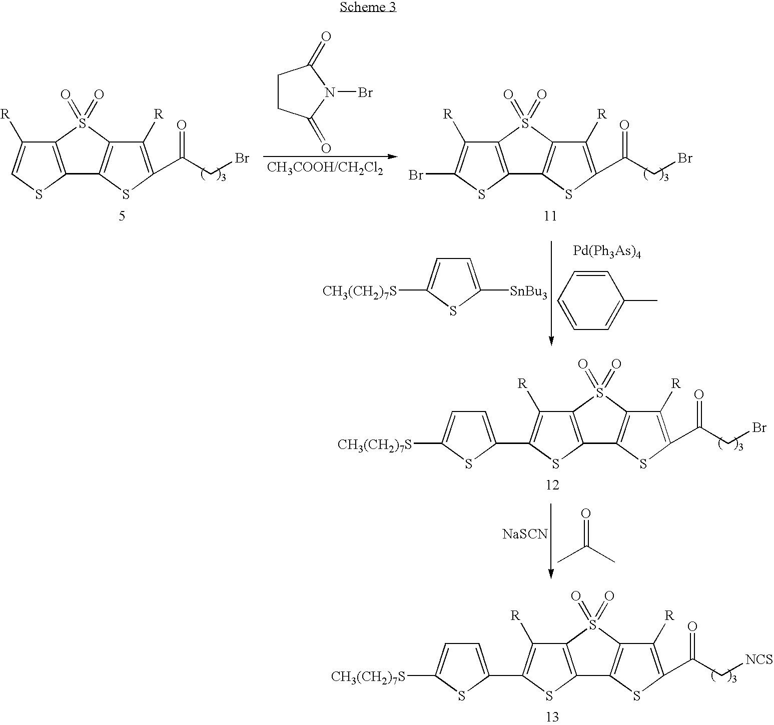

### EXAMPLE 11

### 4-bromo-1-(4,4-dioxy-6-bromo-dithien[3,2-b;2′,3′-d]thien-2-yl)-butane-1-one (11)

Starting with 4-bromo-1-(4,4-dioxy)-dithien[3,2-b;2′,3′-d]thien-2-yl-butane-1-one (5) and following a similar procedure to that described in example 8 a yellow solid is obtained: mp 210° C.; MS m/e 456 (M^{+}); λₘₐₓ (CH₂Cl₂)=385 nm; FTIR (neat) v_{SO2}1311, 1156 cm⁻¹, v_{CO} 1651 cm⁻1; ¹H NMR (CDCl₃, TMS/ppm) δ 7.777 (s, 1H), 7.292 (s, 1H), 3.526 (t, ³J=6.4 Hz, 2H), 3.106 (t, ³J=6.8 Hz, 2H), 2.311 (m, 2H); ¹³C NMR (CDCl₃, TMS/ppm) δ 151.137, 141.431, 141.044, 136.331, 132.780, 122.558, 117.223, 115.773, 32.828, 31.545, 29.777, 29.686

### EXAMPLE 12

### 4-bromo-1-(4,4-dioxy-6-(5′-octylsulfanyl-thien-2′-yl)-dithien[3,2-b;2′,3′-d]thien-2-yl)-butane-1-one (12)

Staring with 4-bromo-1-(4,4-dioxy-6-bromo-dithien[3,2-b;2′,3′-d]thien-2-yl)-butane-1-one (11) and following a similar procedure to that described in example 9 a red solid is obtained: mp 155° C.; MS m/e 604 (M^{+}); FTIR (neat) v_{SO2}1306, 1139 cm⁻¹, v_{CO} 1654 cm⁻¹; λₘₐₓ (CH₂Cl₂)=440 nm; λₑₘ (CH₂Cl₂)=600 nm; ε (CH₂Cl₂)=14187 mol⁻¹*cm⁻1, ¹H NMR (CDCl₃, TMS/ppm) δ 7.284 (d, ³J=3.6 Hz, 2H), 7.246 (d, ³J=3.6 Hz, 2H), 7.245 (s, 2H), 7.140 (bs, 4H), 2.966 (s, 4H), 0.486 (s, 12H); ¹³C NMR (CDCl₃₁ TMS/ppm) δ 142.894, 142.227, 142.079, 138.053, 136.315, 135.943, 133.947, 133.196, 125.933, 125.758, 124.924, 115.574, 30.327, −3.505

### EXAMPLE 13

### 4-isothiocyanate-1-(4,4-dioxy-6-(5′-octylsulfanyl-thien-2′-yl)-dithien[3,2-b;2′,3′-d]thien-2-yl)-butane-1-one (13)

Starting with 4-bromo-1-(4,4-dioxy-6-(5′-octysulfanyl-thien-2′-yl)-dithien[3,2-b;2′,3′-d]thien-2-yl)-butane-1-one (12) and following a similar procedure to that described in example 10, a red solid is obtained: MS m/e 581 (M^{+}); FTIR (neat) v_{SO2}1305, 1140 cm⁻¹, v_{CO} 1656 cm⁻¹, v_{NCS} 2152 cm⁻¹; λₘₐₓ (CH₂Cl₂)=480 nm; λₑₘ (CH₂Cl₂)=586 nm; ε (CH₂Cl₂)=44480 mol⁻¹*cm⁻¹; ¹H NMR (CDCl₃, TMS/ppm) δ 7.761 (s, 1H), 7.537 (d, ³J=5.2 Hz, 1H), 7.291 (d, ³J=5.2 Hz, 1H), 7.118 (bs, 4H), 2.498 (s, 4H), 0.535 (s, 12H); ¹³C NMR (CDCl₃, TMS/ppm) δ 190.308, 146.771, 145.603, 145.079, 142.377, 142.157, 139.038, 136.564, 133.195, 131.707, 126.092, 123.618, 115.718, 111.681, 38.661, 35.816, 33.069, 31.763, 29.373, 29.145, 29.062, 28.439, 23.924, 22.634, 14.097

### EXAMPLE 14

### 1-(2-[2,2′;5′,2″]Tri thien-5-yl-ethyl)-maleimide

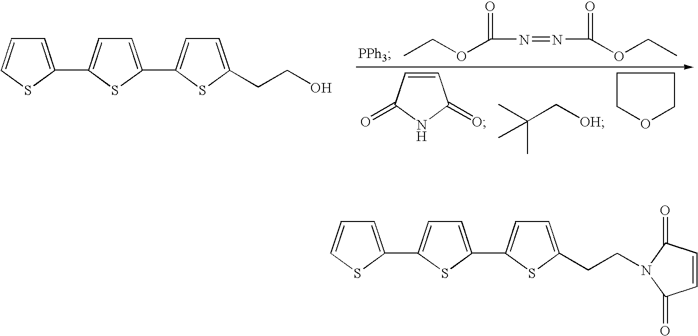

Triphenylphosphine (0.374 mmol, 0.098 g) is placed in a flask under nitrogen in 5 mL of anhydrous THF. The flask is cooled to −78° C. and diethylazodicarboxylate (0.374 mmol, 0.06 mL) is added. It is left to be stirred for 5 min then 2-[2,2′;5′,2″]terthien-5-yl-ethanol (0.34 mmol, 0.100 g) is added leaving it to stir for another 5 min. Neopentyl alcohol (0.175 mmol, 0.015 g) and maleimide (0.374 mmol, 0.037 g) are added. This is left at −78° C. for a further 5 minutes then at room temperature for the whole night. The reaction crude is purified using a chromatographic column on silica gel eluting with petroleum ether: ethyl acetate 7:3. A yellow-orange solid is obtained.

¹H NMR (CDCl₃, TMS/ppm) δ 7.21 (dd, ³J=5.2 Hz, ⁴J=1.4 Hz, 1H), 7.16 (dd, ³J=3.8 Hz, ⁴J=1.2 Hz, 1H), 7.02 (m, 4H), 6.74 (d, ³J=3.6 Hz, 1H), 6.70 (s, 2H), 3.82 (t, 2H), 3.11 (t, 2H); ¹³C NMR (CDCl₃, TMS/ppm) δ 170.42, 139.15, 137.126, 136.10, 135.90, 135.93, 134.15, 127.86, 126.52, 124.42, 124.25, 123.94, 123.63, 123.52, 38.94, 28.70.

### EXAMPLE 15

### Ester 2,5-dioxy-pyrrodilin-1-ylic of 2,2′-bithiophene-5-carboxylic acid

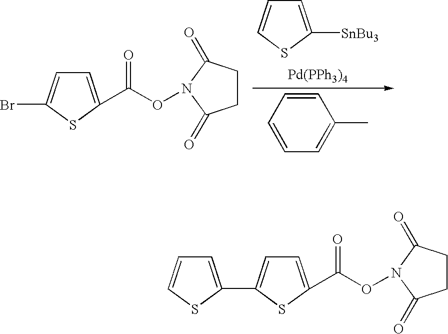

A bromine derivative (0.5 moles) is added to a solution of Pd(PPh₃)₄ prepared in situ (0.015 mmoles) in toluene (5 mL) in an inert atmosphere. The mixture is heated to 80° C., followed by the introduction of stannum derivative (0.5 mmoles) dissolved in toluene (3 mL). After 2 hours the reaction mixture is left to cool at room temperature, the solvent is removed and the compound purified using flash-chromatography (silica gel, petroleum ether-ethyl acetate 1:1) in order to obtain a white microcrystalline solid. Yield: 127 mg (83%), pf 159-160° C.; EI-MS m/z 307 (M⁺); λₘₐₓ (CH₂Cl₂) 347 nm; εₘₐₓ 24000; λₑₘ (CH₂Cl₂) 418 nm; ¹H NMR (CDCl₃, TMS/ppm) δ 7.91 (d, ³J=4.0 Hz, 1H), 7.36 (dd, ³J=5.0 Hz, ⁴J=0.8 Hz, 1H), 7.33 (dd, ³J=3.6 Hz, ⁴J=0.8 Hz, 1H), 7.21 (d, ³J=4.0 Hz, 1H), 7.07 (dd, ³J=3.6 Hz, ³J=5.0 Hz, 1H), 2.89 (s, 4H); ¹³C NMR (CDCl₃, TMS/ppm) δ 169.14, 157.09, 147.71, 137.52, 135.41, 128.32, 127.13, 126.19, 124.26, 124.05, 25.58; Anal. calculated for C₁₃H₉NO₄S₂ (307, 34): C, 50.80; H, 2.95. Found: C, 50.92; H, 3.02

### EXAMPLE 16

ester 2,5-dioxy-pyrrolidin-1ylic of 2,2′;5′,2′-terthiophene-5-carboxylic acid

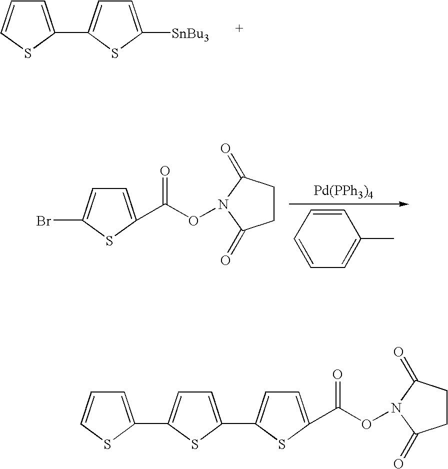

Following a procedure similar to that described in example 15 an amorphous lemon yellow solid is obtained. Yield: 185 mg (95%), pf 223-224° C.; EI-MS m/z 389 (M⁺); m, (CH₂Cl₂) 395 nm; εₘₐₓ 36900; λₑₘ (CH₂Cl₂) 482 nm; ¹H NMR (CDCl₃, TMS/ppm) δ 7.92 (d, ³J=4.0 Hz, 1H), 7.28 (dd, ³J=5.2 Hz, ⁴J=1.2 Hz, 1H), 7.26 (d, ³J=4.0 Hz, 1H), 7.23 (dd, ³J=4.0 Hz, ⁴J=1.2 Hz, 1H), 7.20 (d, ³J=4.0 Hz, 1H), 7.13 (d, ³J=4.0 Hz, 1H), 7.05 (dd, ³J=4.0 Hz, ³J=5.2 Hz, 1H), 2.90 (s, 4H); ¹³C NMR (CDCl₃, TMS/ppm) δ 169.14, 157.08, 147.41, 139.22, 137.61, 136.31, 133.89, 128.07, 126.93, 125.43, 124.63, 124.54, 124.05, 123.95, 25.62; Anal. calculated for C₁₇H₁₁NO₄S₃ (389.47): C, 52.43; H, 2.85; found: C, 52.54; H, 2.98.

### EXAMPLE 17

### Ester 2,5-dioxy-pyrrodilin-1-ylic of 2,2′;5′,2″;5″,2′″-quaterthiophene-5-carboxylic acid

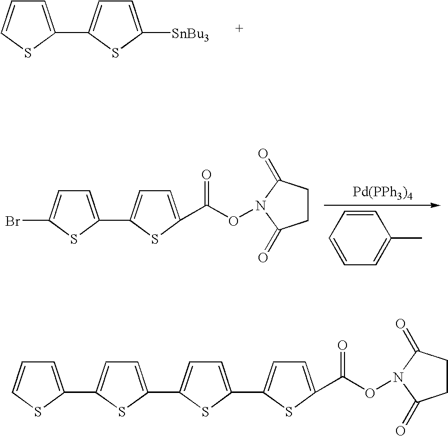

Following a procedure similar to that described in example 15 an amorphous orange coloured solid is obtained. Yield: 188 mg (80%), pf 263-264° C.; EI-MS m/z 471 (M⁺); λₘₐₓ (CH₂Cl₂) 421 nm; εₘₐₓ 45100; λₑₘ (CH₂Cl₂) 536 nm; ¹H NMR (CDCl₃, TMS/ppm) δ 7.93 (d, ³J=4.0 Hz, 1H), 7.26 (d, ³J=4.0 Hz, 1H), 7.24 (dd, ³J=4.0 Hz, ⁴J=1.2 Hz, 1H), 7.21 (d, ³J=4.0 Hz, 1H), 7.20 (dd, ³J=5.2 Hz, ⁴J=1.2 Hz, 1H), 7.12 (m, 3H), 7.04 (dd, ³J=4.0, ³J=5.2, 1H), 2.90 (s, 4H); ¹³C NMR (CDCl₃, TMS/ppm) δ 169.10, 157.08, 147.32, 138.93, 137.60, 137.45, 137.39, 136.76, 134.97, 133.97, 127.97, 127.02, 125.17, 124.91, 124.53, 124.49, 124.10, 124.07, 25.64; Anal. calculated for C₂₇H₂₅NO₇S₄ (4713.59): C, 53.48; H, 2.78. Found: C, 53.54; H, 2.83.

### EXAMPLE 18

### Ester 2,5-dioxy-pyrrodilin-2-ylic of 5′-[2-(2-methoxy-ethoxymethoxy)-ethyl]-[2,2′]bithiophene-5-carboxylic acid

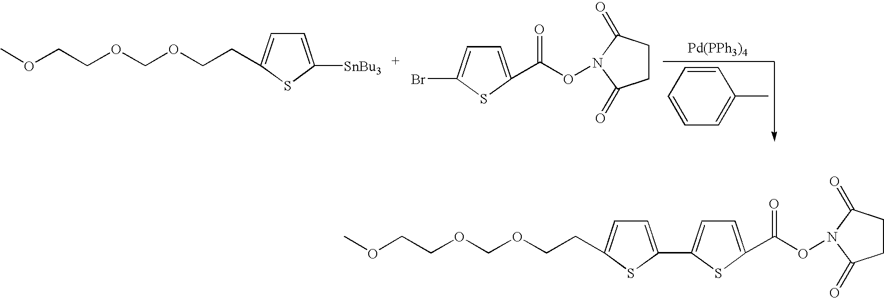

Following a procedure similar to that described in example 15 a light yellow oil is obtained. Yield; 180-mg (82%), EI-MS m/z 439 (M⁺); λₘₐₓ (CH₂Cl₂) 359 nm; δₘₐₓ 26700; λₑₘ (CH₂Cl₂) 432 nm; ¹H NMR (CDCl₃, TMS/ppm) δ 7.89 (d, ³J=4.0 Hz, 1H), 7.15 (d, ³J=4.0 Hz, 1H), 7.12 (d, ³J=4.0 Hz, 1H), 6.80 (d, ³J=4.0 Hz, 1H), 4.75 (s, 2H), 3.81 (t, J=6.0 Hz, 2H), 3.67 (m, 2H), 3.53 (m, 2H), 3.37 (s, 3H), 3.09 (t, J=6.0 Hz, 2H), 2.88 (s, 4H); ¹³C NMR (CDCl₃, TMS/ppm) δ 169.14, 157.06, 148.09, 144.12, 137.48, 133.67, 126.57, 125.91, 123.59, 123.41, 95.43, 71.61, 67.70, 66.85, 58.89, 30.70, 25.52; Anal. calculated for C₁₉H₂₁NO₇S₂ (439.50): C, 51.92; H, 4.82. Found: C, 51.95; H, 4.96.

### EXAMPLE 19

### Ester 2,5-dioxy-pyrrodilin-1-ylic of 5″-[2-(2-methoxy-ethoxymethoxy)-ethyl]-[2,2′;5′,2″]terthiophene-5-carboxylic acid

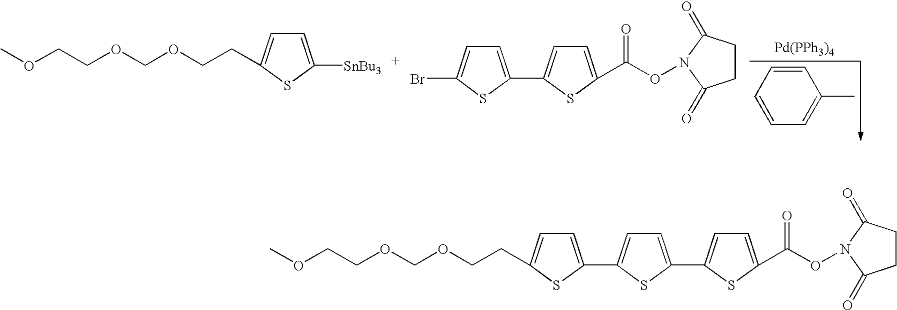

Following a procedure similar to that described in example 15 a yellow-orange poly-crystalline solid is obtained. Yield: 209 mg (80%), pf 119-120° C.; EI-MS m/z 521 (M⁺); λₘₐₓ (CH₂Cl₂) 404 nm; εₘₐₓ 33700; λₑₘ (CH₂Cl₂) 498 nm; ¹H NMR (CDCl₃, TMS/ppm) δ 7.88 (d, ³J=4.0 Hz, 1H), 7.21 (d, ³J=4.0 Hz, 1H), 7.16 (d, ³J=4.0 Hz, 1H), 7.02 (d, ³J=4.0 Hz, 2H), 6.77 (d, ³J=3.6 Hz, 1H), 4.74 (s, 2H), 3.80 (t, J=6.2 Hz, 2H), 3.66 (m, 2H), 3.52 (m, 2H), 3.36 (s, 3H), 3.07 (t, J=6.2 Hz, 2H), 2.87 (s, 4H); ¹³C NMR (CDCl₃, TMS/ppm) δ 169.14, 157.02, 147.47, 142.08, 139.54, 137.55, 134.54, 133.32, 126.88, 126.24, 124.17, 123.97, 123.87, 123.70, 95.46, 71.66, 67.91, 66.85, 58.95, 30.70, 25.58; Anal. calculated for C₂₃H₂₃NO₇S₃ (521.63): C, 52.96; H, 4.44. Found: C, 52.98; H, 4.49.

### EXAMPLE 20

### Ester 2,5-dioxy-pyrrodilin-1-ylic of 5′″-[2-(2-methoxy-ethoxymethoxy)-ethyl]-[2,2′;5′,2″;5″,2′″]quaterthiophene-5-carboxylic acid

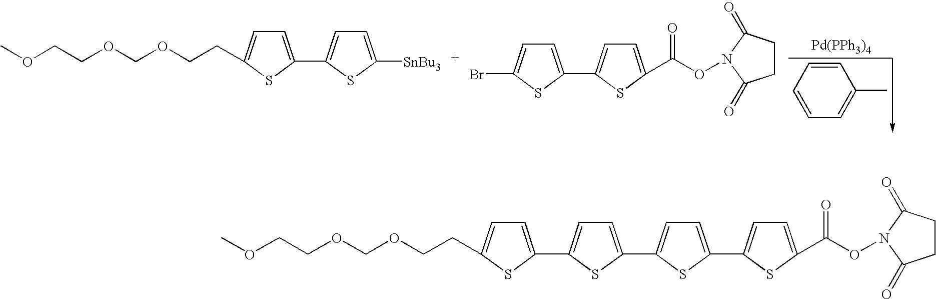

Following a similar procedure to that described in example 15 an amorphous orange coloured solid is obtained. Yield: 260 mg (86%), pf 141-142° C.; EI-MS m/z 603 (M⁺); λₘₐₓ (CH₂Cl₂) 427 nm; εₘₐₓ 61900; λₑₘ (CH₂Cl₂) 552 nm; ¹H NMR (CDCl₃, TMS/ppm) δ 7.91 (d, ³J=4.0 Hz, 1H), 7.24 (d, ³J=4.0 Hz, 1H), 7.19 (d, ³J=4.0 Hz, 1H), 7.09 (m, 2H), 7.01 (m, 2H), 6.77 (d, ³J=4.0, 1H), 4.75 (s, 2H), 3.81 (t, J=6.6 Hz, 2H), 3.68 (m, 2H), 3.54 (m, 2H), 3.38 (s, 3H), 3.08 (t, J=6.6 Hz, 2H), 2.89 (s, 4H); ¹³C NMR (CDCl₃, TMS/ppm) δ 169.10, 157.06, 147.34, 141.52, 139.03, 137.74, 137.59, 135.03, 134.45, 133.79, 127.00, 126.18, 125.13, 124.37, 124.04, 123.98, 123.87, 123.72, 95.55, 71.74, 68.05, 66.92, 59.00, 30.78, 25.63; Anal. calculated for C₂₇H₂₅NO₇S₄ (603.05): C, 53.71; H, 4.17. Found: C, 53.82; H, 4.23.

### EXAMPLE 21

### Ester 2,5-dioxy-pyrrodilin-1-ylic of 5′-methylsulfanyl-[2,2′]bithiophene-5-carboxylic acid

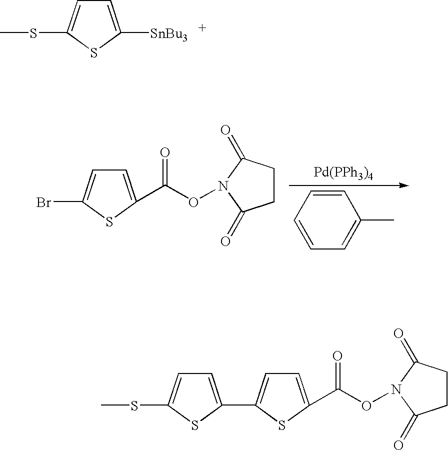

Following a similar procedure to that described in example 15 a lemon yellow microcrystalline solid is obtained. Yield: 143 mg (81%), pf 181-182° C.; EI-MS m/z 353 (M⁺); λₘₐₓ (CH₂Cl₂) 370 nm; εₘₐₓ 27300; λₑₘ (CH₂Cl₂) 479 nm; ¹H NMR (CDCl₃, TMS/ppm) δ 7.90 (d, ³J=4.0 Hz, 1H), 7.18 (d, ³J=4.0 Hz, 1H), 7.15 (d, ³J=4.0 Hz, 1H), 6.99 (d, ³J=4.0 Hz, 1H), 2.90 (s, 4H), 2.55 (B, 3H); ¹³C NMR (CDCl₃, TMS/ppm) δ 169.08, 157.09, 147.21, 140.57, 137.53, 136.60, 130.84, 126.37, 124.08, 124.02, 25.63, 21.44; Anal. calculated for C₁₄H₁₁NO₄S₃ (353.44): C, 47.58; H, 3.14; found: C, 47.76; H, 3.26.

### EXAMPLE 22

### Ester 2,5-dioxy-pyrrodilin-1-ylic of 5-dithien[3,2-b;2′,3′-d]thien-2-yl-thiophene-2-carboxylic acid

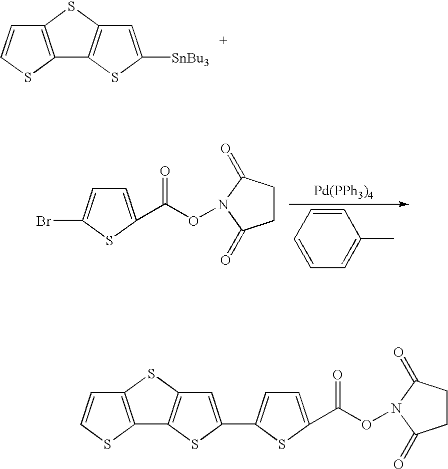

Following a similar procedure to that described in example 15 a yellow-orange amorphous solid is obtained. Yield: 55 mg (26%), pf 239-240° C.; EI-MS m/z 419 (M⁺); λₘₐₓ (CH₂Cl₂) 396 nm; 480 nm; ¹H NMR (CDCl₃, TMS/ppm) δ 7.95 (d, ³J=4.0 Hz, 1H), 7.55 (s, 1H), 7.44 (d, ³J=5.2 Hz, 1H), 7.31 (d, ³J=5.2 Hz, 1H), 7.26 (d, ³J=4.0 Hz, 1H), 2.91 (9, 4H); Anal. calculated for C₁₇H₉NO₄S₄ (418.94): C, 48.67; H, 2.16. Found: C, 48.76; H, 2.22.

### EXAMPLE 23

### Ester 2,5-dioxy-pyrrodilin-1-ylic of 5-dithien[3,2-b;2′,3′-d]thien 4,4-dioxy-2-yl-thiophene-2-carboxylic acid

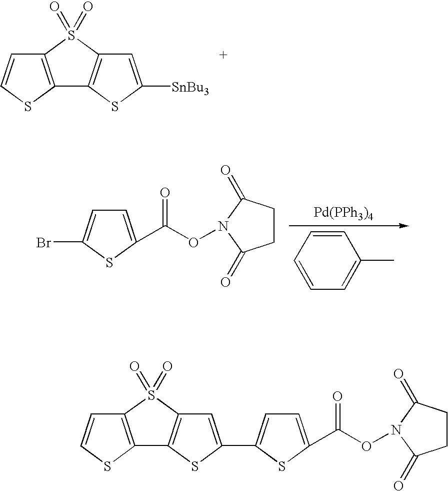

Following a similar procedure to that described in example 15 an orange amorphous solid is obtained. Yield: 140 mg (62%), pf 260-261° C.; EI-MS m/z 307 (M+); λmax (CH₂Cl₂) 405 nm; εₘₐₓ 32400; λem (CH₂Cl₂) 491 nm; ¹H NMR (CDCl₃, TMS/ppm) δ 7.95 (d, 3J=3.6 Hz, 1H), 7.44 (d, 3J=4.8 Hz, 1H), 7.42 (s, 1H), 7.28 (d, 3J=3.6 Hz, 1H), 7.26 (d, 3J=4.8 Hz, 1H), 2.92 (s, 4H); 13C NMR (CDCl3, TMS/ppm) δ 168.95, 156.77, 144.63, 144.05, 143.23, 139.58, 137.47, 135.84, 135.40, 130.75, 126.33, 125.55, 120.56, 118.14, 25.64; Anal. calculated for C17H9NO6S4 (450.93): C, 45.22; H, 2.01. Found: C, 45.25; H, 2.09.

### EXAMPLE 25

### 2-[2-(2-Methoxy-ethoxymethoxy)-ethyl]-thiophene

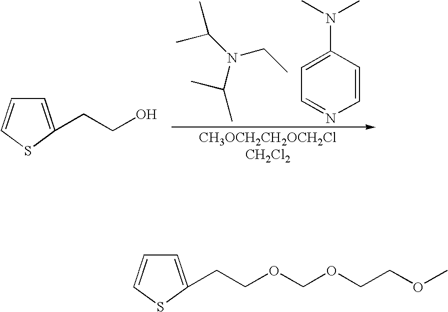

4-dimethylamminopyridine (2%) and methoxyethoxylmethoxyl chloride are added to a mixture of 2-thiophen-2-yl-ethanol (2.56 g 0.02 mol) and N,N-diisopropylethylammine (3.87 g 0.03 mol) dissolved in 30 mL of methylene chloride, a drop at a time at room temperature. The reaction mixture is left to react overnight, then washed several times with a saturated solution of NaHCO₃ and extracted using methylene chloride. The organic phase is anhydrified with sodium sulphate and the solvent removed using the rotavapor. The residue is purified using flash-chromatography on silica eluting with petroleum ether:ethyl acetate 7:3. 4.32 g of yellow oil is obtained (Yield 98%):

MS m/e 141 (M^{+}); ¹H NMR (CDCl₃, TMS/ppm) δ 7.125 (dd, ³J=6.4 Hz, 1H), 6.918 (dd, ³J=8.6 Hz, 1H), 6.846 (m, 1H), 4.732 (B, 1H), 3.796 (t, 2H), 3.654 (m, 2H) 3.529 (m, 2H), 3.373 (8, 3H), 3.106 (t, 2H);

### EXAMPLE 26

The compound

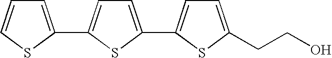

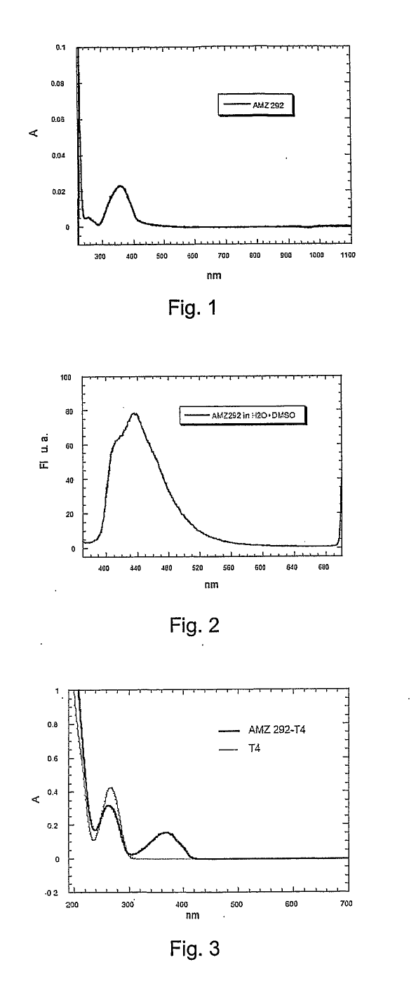

that for simplicity is referred to in this case as A/MZ292, emits in yellow and presents an absorbency spectrum in H₂O/DMSO as shown in FIG. 1, wherein the wave length is shown in nm in the X-axis, and the absorbency is shown in the Y-axis. The absorbency maximum is at a wave length of 356 nm. FIG. 2 shows the UV emission spectrum of A/MZ292 in H₂O/DMSO: wherein the wave length is shown in nm in the X-axis, and the emission intensity is shown in the Y-axis. Starting with A/MZ292 a phosphoroammidite was prepared using the following procedure

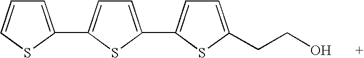

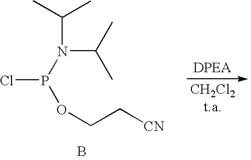

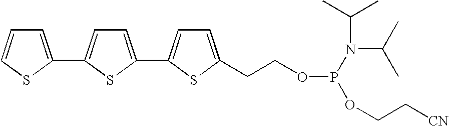

EXPERIMENTAL CONDITIONS

| Reactants | equivalents | PM (d.) | n.moles | g | ml |
|---|---|---|---|---|---|
| A/MZ-292 | 1 | 292 | 0.1506 | 0.044 | |
| B | 2 | 236.68 | 0.3012 | 0.0713 | 0.07 |
| | | (1.061) | | | |
| DIPEA (N,N,- | 5 | 129.25 | 0.753 | 0.0973 | 0.129 |
| diisopropyl- | | (0.757) | | | |
| ethylamine) | | | | | |
| CH₂Cl₂ | | | | | 5.0 |

Process: A/MZ-292 and CH₃CN are introduced into a 50 ml single-necked flask and coevaporated to anhydrify the powder.

Under nitrogen atmosphere, 5 ml of CH₂Cl₂ and 0.129 ml of DIPEA are added to the residue and mixed for 5 minutes; using a syringe, B is added a drop at a time; it is left to react, controlling the reaction progress through TLC (eluent mixture: CH₂Cl₂/EtOAc/Et₃N; 45/45/10).

After 30 minutes the reaction is considered completed and WORK UP is started with a saturated solution of NaHCO₃ and then with water. Anhydrification is carried out with anhydrous Na₂SO₄, followed by solution filtering and concentration.

The purification of the reaction crude occurs through preparative TLC on a plate (eluent mixture: CH₂Cl₂/EtOAc/Et₃N; 45/45/10).

The phosphoroammidite obtained was used for synthesis, on an automatic synthesizer for oligonucleotides, with two conjugates

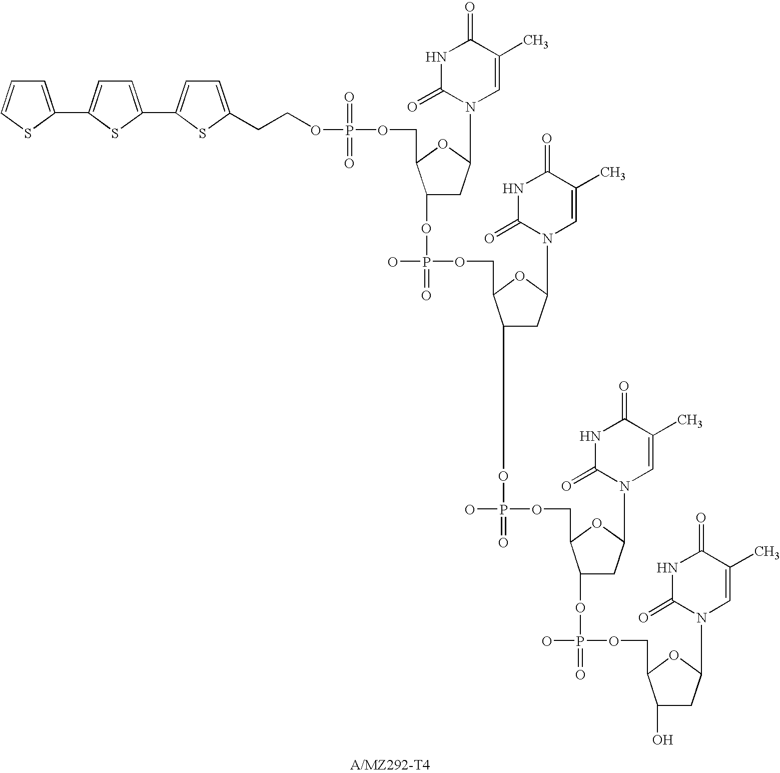

whose absorption profile in H₂O is shown in FIG. 3: the wave length is shown in run in the X-axis and the absorbency is shown in the Y-axis.

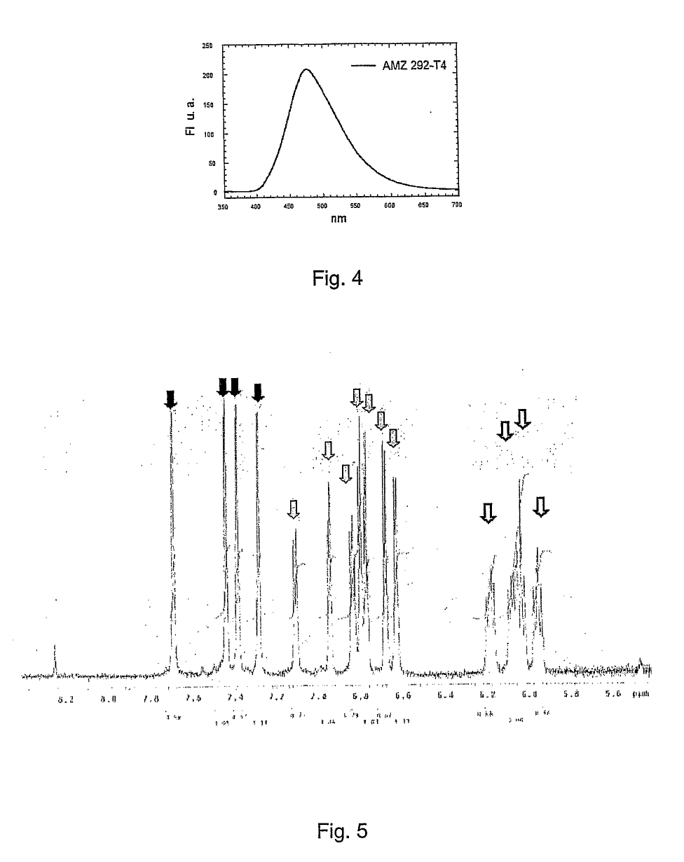

A/MZ292-T4 has an emission profile shown in FIG. 4: the wave length is shown in nm in the X-axis and the emission intensity in H₂O is shown in the Y-axis.

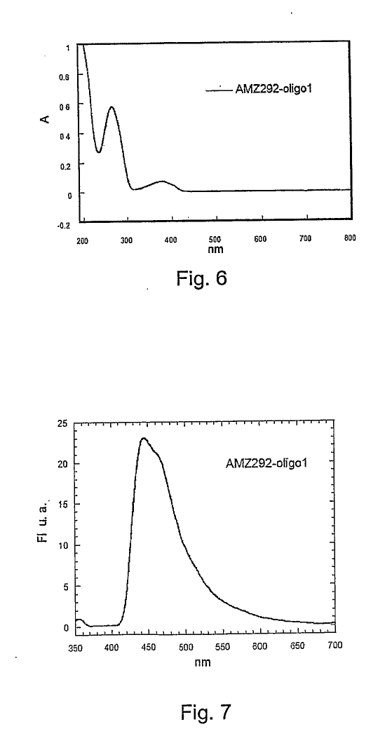

La FIG. 5 shows the NMR spectrum of A/MZ292-T4. The dark arrows represent the four protons in position 6 of the thymines, the light arrows show the oligothiophene protons, and the transparent arrows show the 4 protons in position 1′ of the sugar residues of the oligonucleotide.

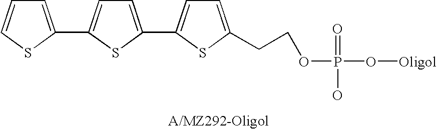

wherein Oligo1 represents 5′ACCACCCTTCGAACCACAC 3′. A/MZ292-Oligol presents the absorption profile shown in FIG. 6 and the emission profile shown in FIG. 7.

### EXAMPLE 27

The compound

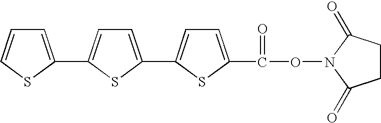

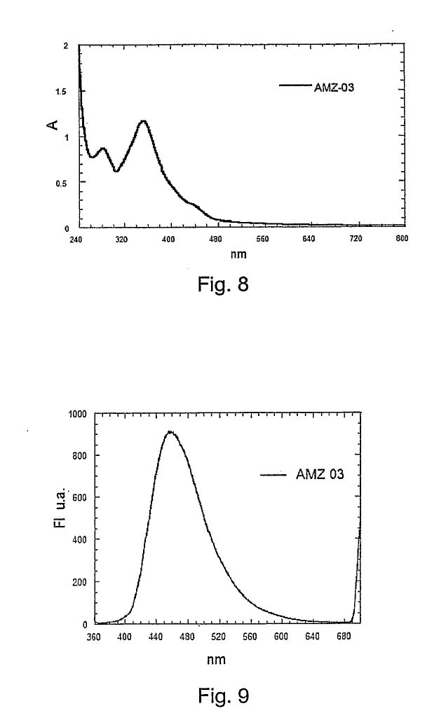

which for simplicity, is referred to in this case as A/MZ03, emits in yellow and presents an absorbency spectrum as shown in FIG. 8, and the emission spectrum as shown in FIG. 9. A/MZO3 was conjugated post-synthesis to an oligonucleotide with the following process:

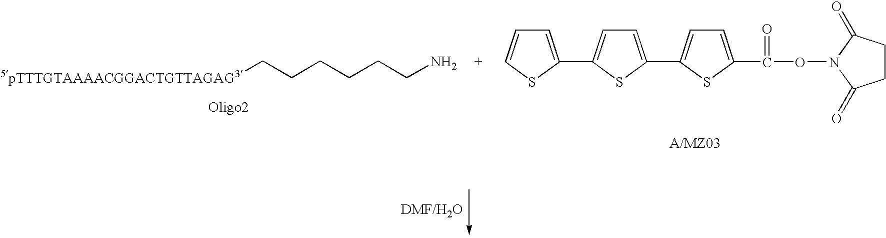

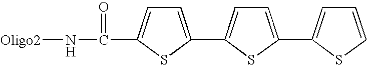

Oligo2-A/MZ03
EXPERIMENTAL CONDITIONS

| Reactants | Quantity | μmol | OD | mg | μl |
|---|---|---|---|---|---|
| Oligo2 | 1 eq. | 0.0486 | 10.86 | | |
| A/MZ-03 | 1 mg for each 10 OD of Oligo2 | | | 1.0 | |
| H₂O | 250 μl for each μmol of Oligo2 | | | | 12.2 |
| DMF | μl H₂O × 5 | | | | 60.8 |

Process: Oligo2 is dissolved in water, A/MZ-03 and DMF are added. Since the fluorescent compound does not dissolve, a further 60.8 μl of DMF is added. The sample is placed in an oven at 46° C. for 16 h. It is controlled to ensure that conjugation has occurred by means of injection in HPLC in inverse phase.

The excess A/MZ-03 is extracted using a mixture of CH₂Cl₂/MeOH (85/15) until the organic phase remains without any color. The conjugated Oligo2-A/MZ03 extracted in water emits fluorescence in yellow when irradiated at 360 nm.

1. Oligonucleotide probe presenting a general formula (I):
Onu-L-Thioₙ (I)
wherein Onu represents an oligonucleotide residue;
independently from the other Thio, each Thio represents, a respective thiophenic ring; Thioₙ represents a fluorescent oligothiophene containing n thiophenic rings;
n is an integer lower than eight;
L represents a binder conceived to maintain Thioₙ mobile in relation to Onu so that Thioₙ is able to perform its fluorescent action correctly and Onu is able to hybridize freely with a complementary sequence;
with the proviso that if n is equal to 1, the sulphur of the thiophenic ring is bonded to two oxygens.

2. Probe according to claim 1, wherein
independently from the other Thio, each Thio represents, a respective thiophenic ring presenting the general formula II: 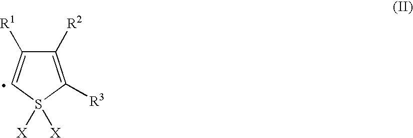
wherein X is selected from a free lone pair of electrons of S and an oxygen radical; R¹, R², R³, each independently from one other, are selected from the group consisting of: hydrogen, halogen, alkyl C₁-C₁₂, aromatic, —CN, —NO₂, -Thioₘ, —NR⁴R⁵, —SR⁴, —R⁵—SR⁴, —OR⁴, —R⁵—OR⁴, —C(O)R⁶, —NC(O)R⁶, —O—PG, —R⁷—O—PG; wherein R⁴ and R⁵ are selected, each independently from each other, from the group composed of hydrogen, alkyl C₁-C₁₂, aromatic, Thioₘ; R⁶ is selected from the group consisting of hydrogen, halogen, alkyl C₁-C₁₂, aromatic, Thioₘ; m is an integer lower than n; —PG is a protecting group of hydroxy that is removable in acidic environments; with the proviso that if n is equal to 1× represents an oxygen radical; each Thio group can be fused with 1 or 2 other Thio groups; R⁷ is a saturated alkylene C₁-C₈.

3-52. (canceled)

53. Probe according to claim 1, wherein L is selected from the group consisting of: alkylene C₂-C₈, —R⁸—C(O)—, —R⁸—O—C(O)—, —R⁸—C(O)—R⁹—, —R⁸—O—C(O)—R⁹—, —R⁸—O—R⁹—C(O)—, —R⁸—O—R⁸—, —R⁸—S—, —R⁸—S—R⁹—C(O)—, —R⁸—S—R¹²—, —R⁸—NH—R⁹—C(O)—, —R⁸—NH—R¹²—, —R⁸—Si(R¹⁰)(R¹¹)—, —O—P(O)₂—O—R¹²—, —O—P(O)₂—O—R⁹—C(O)—, —R⁸—NH—(O)C—R⁹—C(O)—, —R⁸—NH—(O)C—R¹²—, —R⁸—NH—(O)C—, —R⁸—NH—C(S)—NH—R¹²—, —R⁸—NH—C(S)—NH—R⁹—C(ON, 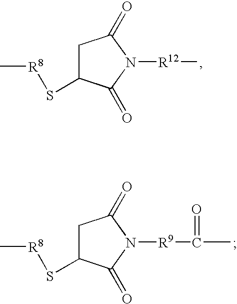
wherein R⁸ and R⁹ represent, each independently from each other, a respective alkylene C₁-C₈; R¹² is selected from the group consisting of: an alkylene C₁-C₉, —R⁹—Si(R¹⁰)(R¹¹)—; R¹⁰ and R¹¹ being selected, each independently from each other, from the group consisting of: alkyl C₁-C₈, aryl, alkenyl C₂-C₈, alkynyl C₂-C₈.

54. Probe according to claim 53, wherein L is selected from the group consisting of: saturated alkylene C2-C8, —R8-C(O)—, —R8-O—C(O)—, —R8-C(O)—R9-, —R8-O—C(O)—R9-, —R8-O—R9-C(O)—, —R8-O—R12-, —R8-S—R9-C(O)—, —R8-S—R12-, —R8-NH—R9-C(O)—, —R8-NH—R12-, —R8-Si(R10)(R11)-, —O—P(O)2-O—R12-, —O—P(O)2-O—R9-C(O)—, —R8-NH—(O)C—R9-C(O)—, —R8-NH—(O)C—R12-, —R8-NH—(O)C—, —R8-NH—C(S)—NH—R12-, —R8-NH—C(S)—NH—R9-C(O)—, 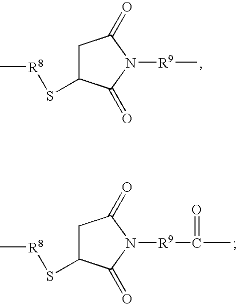
wherein R⁸ and R⁹ represent, each independently from each other, a respective saturated alkylene C₁-C₈; R¹⁰ and R¹¹ represent, each independently from each other, a respective alkyl C₁-C₆; R¹² is selected from the group consisting of an alkylene C₂-C₉, —R⁹—Si(R¹⁰)(R¹¹)—.

55. Probe according to claim 54, wherein R⁸ and R⁹ represent, each independently from each other, a respective saturated alkylene C₁-C₆; R¹² represents a saturated alkylene C₂-C₇.

56. Probe according to claim 53, wherein L is selected from the group consisting of: —R⁸—S—R⁹—C(O)—, —R⁸—S—R¹²—, —O—P(O)₂—O—R², —O—P(O)₂—O—R⁹—C(O)— —R⁸—NH—(O)C—R⁹—C(O)—, —R⁸—NH—(O)C—R¹²—, —R⁸—NH— (O)C—, —R⁸—NH—C(S)—NH—R¹², —R⁸—NH—C(S)—NH—R⁹—C(O)—,

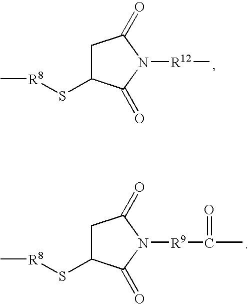

57. Probe according to claim 53, wherein L is selected from the group consisting of: —O—P(O)₂—O—R¹²—, —O—P(O)₂—O—R⁹—C(O)—, —R⁸—NH—(O)C—R⁹—C(O)—, —R⁸—NH—(O)C—R¹²—, —R⁸—NH— (O)C—;

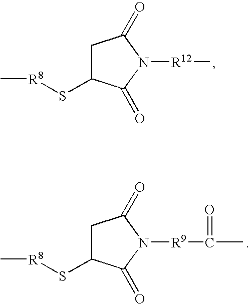

58. Probe according to claim 57, wherein L is selected from the group consisting of: —R⁸—NH—(O)C—, —O—P(O)₂—O—R¹²—.

59. Probe according to claim 53, wherein R⁸ and R⁹ represent, each independently from each other, a saturated linear alkylene C₁-C₆ and R¹² represents a saturated linear alkylene C₂-C₃.

60. Probe according to claim 2, wherein n is lower than 5;
R¹, R², R³, each independently from one other, being selected from the group consisting of: alkyl C₁-C₈, —R⁵—S—R⁴, hydrogen, —SR⁴, —R⁷—O—PG; wherein R⁷, R⁴ and R⁵ represent, each independently from one other, a saturated alkylene C₁-C₈; —PG and a protecting group of an hydroxy group that is removable in an acidic environment.

61. Probe according to claim 60, wherein R¹, R², R³ each independently from one other, are selected from the group consisting of: hydrogen, —SR⁴, —R⁷—O—PG.

62. Probe according to claim 61, wherein R¹, R², R³ each represent a respective hydrogen.

63. Probe according to claim 2, wherein X represents a lone pair of electrons of S.

64. Probe according to claim 1, wherein Thioₙ represents:

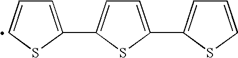

65. Probe according to claim 1, wherein —PG is selected from the group consisting of: dimethoxytrityl, monomethoxytrityl, methoxy ethoxy methyl (MEM), methoxy methyl (MOM).

66. Beacon compound having a general formula III: 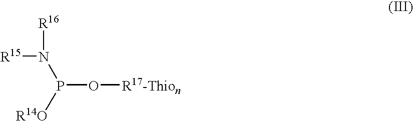
wherein R¹⁴ is a protecting group of the phosphite ester and is removable through the action of aqueous ammonia at 30%;
R¹⁵ and R¹⁶ being chosen so that NR¹⁵R¹⁶ is removable through the action of weak acids, in particular tetrazole;
independently from the other Thio, each Thio represents, a respective thiophenic ring; Thioₙ represents a fluorescent oligothiophene containing n thiophenic rings;
R¹⁷ being selected from the group consisting of: —R¹²— and —R⁹—C(O)—;
R¹² being selected from the group consisting of: an alkylene C₁-C₉, —R⁹—Si(R¹⁰)(R¹¹)—; R¹⁰ and
R¹¹ being selected, each independently from each other, from the group consisting of: alkyl C₁-C₆, aryl, alkenyl C₂-C₈, alkynyl C₂-C₈;
R⁹ representing an alkylene C₁-C₈.

67. Compound according to claim 66, wherein Thioₙ is defined according to claim 2;
R¹² representing a saturated alkylene C₂-C₇;
R⁹ representing a saturated alkylene C₁-C₆.

68. Compound according to claim 66, wherein Thioₙ is defined according to claim 60.

69. Compound according to claim 66, wherein R¹⁷ represents a saturated linear alkylene C₂-C₃.

70. Compound according to claim 66, wherein R¹⁵ and R¹⁶ are selected, each independently from each other, from the group consisting of: alkyl C₁-C₁₂, aryl, cycloalkyl C₅-C₁₀; as an alternative option, R¹⁵ and R¹⁶ can be linked in a manner to form with N, a heterocyclic 5-6 member ring.

71. Compound according to claim 66, wherein R¹⁴ is selected from the group consisting of: —(CH₂)₂CN and —CH₃.

72. Compound according to claim 66, wherein R¹⁵ and R¹⁶ represent, each independently from each other, a saturated alkyl C₁-C₃.

73. Compound according to claim 72, wherein R¹⁵ and R¹⁶ represent, each independently from each other, a saturated alkyl C₃.

74. Compound according to claim 73, wherein R¹⁵ and R¹⁶ each represent a respective isopropyl group.

75. Method for the preparation of an oligonucleotide probe having general formula I according to claim 1, wherein L is selected from the group consisting of —O—P(O)₂—O—R¹²—, —O—P(O)₂—O—R⁹—C(O)—; the method involving a conjugation phase, wherein a compound having a general formula III according to claim 66 is bonded to an oxygen in position 5′ of a nucleoside of an Onu oligonucleotide residue; during said conjugation phase, NR¹⁵R¹⁶ being removed and P being oxidized; a removal phase, that occurs in basic conditions and during which R¹⁴ is removed.

76. Method according to claim 75, wherein the conjugation phase occurs in a moderately acidic environment in particular in the presence of tetrazole.

77. Method according to claim 75, the conjugation phase being carried out in the typical conditions of an automatic oligonucleotide synthesizer.

78. Method according to claim 75, and including a nucleophilic substitution phase, wherein a first reagent, which presents a general formula Thioₙ-R¹⁷—O⁻, is made to react with a second reagent having the general formula IV: 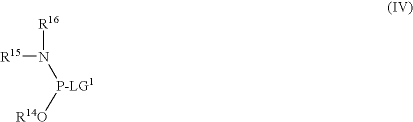
in order to obtain the compound presenting the general formula III according to claim 16;
LG¹ being a leaving group.

79. Method according to claim 78, wherein LG¹ is a leaving group selected from the group consisting of: halogen and NR¹⁵R¹⁶.

80. Beacon compound having a general formula V: 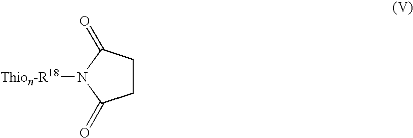
independently from the other Thio, each Thio represents, a respective thiophenic ring; Thioₙ represents a fluorescent oligothiophene containing n thiophenic rings;
R¹⁸ being selected from the group consisting of: —C(O)—O—, —R¹²—C(O)—O—, —C(O)—R⁹—C(O)—O—;
R¹² being selected from the group consisting of: an alkylene C₁-C₉, —R⁹—Si(R¹⁰)(R¹¹)—; R¹⁰ and
R¹¹ being selected, each independently from each other, from the group consisting of: alkyl C₁-C₆, aryl, alkenyl C₂-C₈, alkynyl C₂-C₈;
R⁹ representing an alkylene C₁-C₈.

81. Compound according to claim 80, wherein Thioₙ is defined according to claim 2.

82. Compound according to claim 80, wherein R¹² and R⁹ are defined, each independently from each other, according to claim 54.

83. Compound according to claim 80, presenting the following formula:

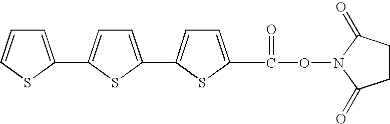

84. Method for the synthesis of an oligonucleotide probe having the general formula I according to claim 1, wherein L is selected from the group consisting of R⁸—NH—(O)C—R⁹—C(O)—, —R⁸—NH—(O)C—R¹²—, —R⁸—NH—(O)C—; the method comprising a conjugation phase wherein a compound having a general formula V according to claim 80 to 33 is made to react with a prearranged oligonucleotide having the formula Onu-R⁸—NH₂, wherein Onu represents an oligonucleotide residue;
R⁸ representing an alkylene C₁-C₈.

85. Method according to claim 84, wherein R⁸ is defined according to claim 4.

86. Method according to claim 84, wherein the compound with which a prearranged oligonucleotide residue is made to react, has a formula:

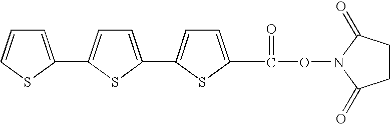

87. Method according to claim 84, wherein R⁸ is a hexyl group.

88. Beacon compound having a general formula VI: 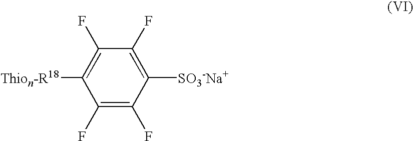
independently from the other Thio, each Thio represents, a respective thiophenic ring; Thioₙ represents a fluorescent oligothiophene containing n thiophenic rings;
R¹⁸ being selected from the group consisting of: —C(O)—O—, —R¹²—C(O)—O—, —C(O)—R⁹—C(O)—O—;
R¹² being selected from the group consisting of: an alkylene C₁-C₉, —R⁹—Si(R¹⁰)(R¹¹)—; R¹⁰ and R¹¹ being selected, each independently from each other, from the group consisting of: alkyl C₁-C₆, aryl, alkenyl C₂-C₈, alkynyl C₂-C₈;
R⁹ representing an alkylene C₁-C₈.

89. Compound according to claim 88, wherein Thioₙ is defined according to claim 2.

90. Compound according to claim 88, wherein R¹² and R⁹ are defined, each independently from each other, according to claim 54.

91. Compound according to claim 88, presenting the following formula:

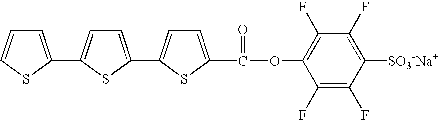

92. Method for the synthesis of an oligonucleotide probe presenting the general formula I according to claim 1, wherein L is selected from the group consisting of R⁸—NH—(O)C—R⁹—C(O)—, —R⁸—NH—(O)C—R¹²—, —R⁸—NH—(O)C—; the method comprising a conjugation phase wherein a compound having the general formula VI according to claim 88 is made to react with a prearranged oligonucleotide residue having the formula Onu-R⁸—NH₂, wherein Onu represents an oligonucleotide residue;
R⁸ representing an alkylene C₁-C₈.

93. Method according to claim 92, wherein R⁸ is defined according to claim 54.

94. Method according to claim 92, wherein the molecule with which a prearranged oligonucleotide residue is made to react, has the formula:

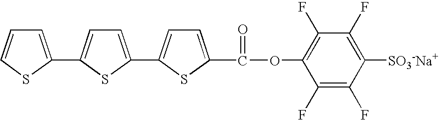

95. Method according to claim 92, wherein R⁸ is a hexyl group.

96. Beacon compound having a general formula VII: 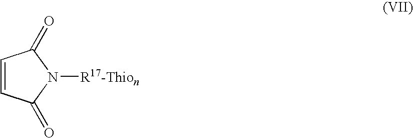
independently from the other Thio, each Thio represents, a respective thiophenic ring; Thioₙ represents a fluorescent oligothiophene containing n thiophenic rings;
R¹⁷ being selected from the group consisting of: —R¹²-and-R⁹—C(O)—;
R¹² being selected from the group consisting of: an alkylene C₁-C₉, —R⁹—Si(R¹⁰)(R¹¹)—; R¹⁰ and R¹¹ being selected, each independently from each other, from the group consisting of: alkyl C₁-C₆, aryl, alkenyl C₂-C₈, alkynyl C₂-C₈; R⁹ representing an alkylene C₁-C₈.

97. Compound according to claim 96, wherein Thio, is defined according to claim 2;
R¹² representing a saturated alkylene C₂-C₉;
R⁹ representing a saturated alkylene C₁-C₈.

98. Compound according to claim 96, wherein Thioₙ is defined according to claim 60.

99. Compound according to claim 96, wherein R¹⁷ represents a saturated alkylene C₂-C₃.

100. Method for the preparation of an oligonucleotide probe having the general formula I according to claim 1, wherein L is selected from the group consisting of 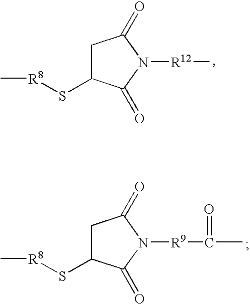
the method involving a conjugation phase, wherein a compound having a general formula VII according to claim 96 is made to react with a prearranged oligonucleotide residue having the formula Onu-R⁸—SH, wherein Onu represents an oligonucleotide residue;
R⁸ representing an alkylene C₁-C₈.

101. Method according to claim 100, and comprising a nucleophilic substitution phase, wherein a first reagent, which is selected from the group consisting of Thio, —R¹²—O⁻ Thioₙ-C(O)—R⁹—O⁻, is made to react with 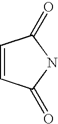
in order to obtain the compound having a general formula VII according to claim 96.

102. Method according to claim 101, wherein R⁸ is defined according to claim 54.
